SEQUENCE LISTING (89 sequences)

SEQ ID NO: 1 - DNA, 40 bp, Artificial Sequence
  Synthetic - dR1
ggtgtctgtgccggtagaggcacagacaccaaaaccaaaa

SEQ ID NO: 2 - DNA, 41 bp, Artificial Sequence
  Synthetic - dR2
aggtgtctgtgccggtagaggcacagacaccaaaaccaaaa

SEQ ID NO: 3 - DNA, 37 bp, Artificial Sequence
  Synthetic - dR3
ggtcaagaggcccatggcctcttgaccaaaaccaaaa

SEQ ID NO: 4 - DNA, 40 bp, Artificial Sequence
  Synthetic - dR4
ggtcaagaggccagctcaggcctcttgaccaaaaccaaaa

SEQ ID NO: 5 - DNA, 52 bp, Artificial Sequence
  Synthetic - dR5
tataaatcttccggttcttccgcctcttccggcggaagaaccaaaaccaaaa

SEQ ID NO: 6 - DNA, 37 bp, Artificial Sequence
  Synthetic - dR6
ggtgtctgtgcctatggcacagacaccaaaaccaaaa

SEQ ID NO: 7 - DNA, 40 bp, Artificial Sequence
  Synthetic - dR7
cacaatcataccagctcaggtatgattgtgaaaaccaaaa

SEQ ID NO: 8 - DNA, 46 bp, Artificial Sequence
  Synthetic - dR8
tcttccggttcttccgccggtagaggcggaagaaccaaaaccaaaa

SEQ ID NO: 9 - DNA, 52 bp, Artificial Sequence
  Synthetic - dR9
taacaatcttccccgtcttccaggtcttcccctggaagacggaaaaccaaaa

SEQ ID NO: 10 - DNA, 41 bp, Artificial Sequence
  Synthetic - dR10
cggtgtctgtgccggtagaggcacagacaccaaaaccaaaa

SEQ ID NO: 11 - DNA, 40 bp, Artificial Sequence
  Synthetic - dR11
ggtgtctgtgccggtagaggcaaccacaccaaaaccaaaa

SEQ ID NO: 12 - DNA, 46 bp, Artificial Sequence
  Synthetic - dR12
tcttccggtgtctgtgccggtagaggcacagacaccaaaaccaaaa

SEQ ID NO: 13 - DNA, 46 bp, Artificial Sequence
  Synthetic - dR13
atgagcggtcaagaggcctcttccggcctcttgaccaaaaccaaaa

SEQ ID NO: 14 - DNA, 43 bp, Artificial Sequence
  Synthetic - dR14
ccaggtgtctgtgccggtagaggcacagacaccaaaaccaaaa

SEQ ID NO: 15 - DNA, 28 bp, Artificial Sequence
  Synthetic - dR15
gtcgacggtagagtcgacaaaaccaaaa

SEQ ID NO: 16 - DNA, 46 bp, Artificial Sequence
  Synthetic - dR16
tcttccggtgtctgtgcctcttccggcacagacaccaaaaccaaaa

SEQ ID NO: 17 - DNA, 52 bp, Artificial Sequence
  Synthetic - dR17
ataaaatcttccgggtcttccgtctcttccgacggaagacccaaaaccaaaa

SEQ ID NO: 18 - DNA, 52 bp, Artificial Sequence
  Synthetic - dR18
aagcctgatccaggtgtctgtgccggtagaggcacagacaccaaaaccaaaa

SEQ ID NO: 19 - DNA, 28 bp, Artificial Sequence
  Synthetic - dR19
gtgcacagctcagtgcacaaaaccaaaa

SEQ ID NO: 20 - DNA, 46 bp, Artificial Sequence
  Synthetic - dR20
tcttccggttcttccgcctcttccggcggaagaaccaaaaccaaaa

SEQ ID NO: 21 - DNA, 46 bp, Artificial Sequence
  Synthetic - dR21
atgagcggttcttccgccagctcaggcggaagaaccaaaaccaaaa

SEQ ID NO: 22 - DNA, 40 bp, Artificial Sequence
  Synthetic - dR22
cagtcattagacggtagagtctaatgactgaaaaccaaaa

SEQ ID NO: 23 - DNA, 46 bp, Artificial Sequence
  Synthetic - dR23
gatccaggtgtctgtgcctcttccggcacagacaccaaaaccaaaa

SEQ ID NO: 24 - DNA, 46 bp, Artificial Sequence
  Synthetic - dR24
tcttccggtcaagaggccagctcaggcctcttgaccaaaaccaaaa

SEQ ID NO: 25 - DNA, 46 bp, Artificial Sequence
  Synthetic - dR25
atgagcggtcaagaggccagctcaggcctcttgaccaaaaccaaaa

SEQ ID NO: 26 - DNA, 40 bp, Artificial Sequence
  Synthetic - dR26
ggtcaagaggccagctcaggccagatgaccaaaaccaaaa

SEQ ID NO: 27 - DNA, 52 bp, Artificial Sequence
  Synthetic - dR27
gcctcgggctcctcatctggtagaagatgaggagcccgaggcaaaaccaaaa

SEQ ID NO: 28 - DNA, 40 bp, Artificial Sequence
  Synthetic - dR28
cggaccacctgccagacagcaggtggtccgaaaaccaaaa

SEQ ID NO: 29 - DNA, 40 bp, Artificial Sequence
  Synthetic - dR29
acgtaatcacatggtagaatgtgattacgtaaaaccaaaa

SEQ ID NO: 30 - DNA, 46 bp, Artificial Sequence
  Synthetic - dR30
gatccaggttcttccgcctcttccggcggaagaaccaaaaccaaaa

SEQ ID NO: 31 - DNA, 46 bp, Artificial Sequence
  Synthetic - dR31
ggtgtctgtgccattatcagcctcggcacagacaccaaaaccaaaa

SEQ ID NO: 32 - DNA, 40 bp, Artificial Sequence
  Synthetic - dR32
ggcggcggggccggtagaggccccgccgccaaaaccaaaa

SEQ ID NO: 33 - DNA, 40 bp, Artificial Sequence
  Synthetic - dR33
cgctgcggtgcgggtagacgcaccgcagcgaaaaccaaaa

SEQ ID NO: 34 - DNA, 46 bp, Artificial Sequence
  Synthetic - dR34
gatccaggtgtctgtgccggtagaggcacagacaccaaaaccaaaa

SEQ ID NO: 35 - DNA, 52 bp, Artificial Sequence
  Synthetic - dR35
ggtgtctgtgccatcactcccatctctccaggcacagacaccaaaaccaaaa

SEQ ID NO: 36 - DNA, 34 bp, Artificial Sequence
  Synthetic - dR36
tcttccaactcttccaaatcttccaaaaccaaaa

SEQ ID NO: 37 - DNA, 46 bp, Artificial Sequence
  Synthetic - dR37
gatccaggttcttccgccggtagaggcggaagaaccaaaaccaaaa

SEQ ID NO: 38 - DNA, 22 bp, Artificial Sequence
  Synthetic - dR38
gacggtagagtcaaaaccaaaa

SEQ ID NO: 39 - DNA, 40 bp, Artificial Sequence
  Synthetic - dR39
acatacatgagtggtagaactcatgtatgtaaaaccaaaa

SEQ ID NO: 40 - DNA, 22 bp, Artificial Sequence
  Synthetic - dR40
ggcggtagagccaaaaccaaaa

SEQ ID NO: 41 - DNA, 40 bp, Artificial Sequence
  Synthetic - dR41
ggctgcgggaccggtagaggtcccgcagccaaaaccaaaa

SEQ ID NO: 42 - DNA, 52 bp, Artificial Sequence
  Synthetic - dR42
gctcctgcaaatttatggagctcaccataaatttgcaggagcaaaaccaaaa

SEQ ID NO: 43 - DNA, 24 bp, Artificial Sequence
  Synthetic - dR43
acatcagacaatgtaaaaccaaaa

SEQ ID NO: 44 - DNA, 52 bp, Artificial Sequence
  Synthetic - dR44
cggaccacctgcaacctaaatccaatcccggcaggtggtccgaaaaccaaaa

SEQ ID NO: 45 - DNA, 46 bp, Artificial Sequence
  Synthetic - dR45
ggtgtctgtgcctttatcacccttggcacagacaccaaaaccaaaa

SEQ ID NO: 46 - DNA, 56 bp, Artificial Sequence
  Synthetic - dR46
cgcggagaccactcggggtccagacagaccccgagtggtctccgcgaaaaccaaaa

SEQ ID NO: 47 - DNA, 46 bp, Artificial Sequence
  Synthetic - dR47
caaaaaggtgtctgtgccggtagaggcacagacaccaaaaccaaaa

SEQ ID NO: 48 - DNA, 24 bp, Artificial Sequence
  Synthetic - dR48
acgccagacagcgtaaaaccaaaa

SEQ ID NO: 49 - DNA, 40 bp, Artificial Sequence
  Synthetic - dR49
gtatttatattcggtagagaatataaatacaaaaccaaaa

SEQ ID NO: 50 - DNA, 52 bp, Artificial Sequence
  Synthetic - dR50
actattatactgaacctaaatccaatcccgcagtataatagtaaaaccaaaa

SEQ ID NO: 51 - DNA, 76 bp, Artificial Sequence
  Synthetic - dR51
ggggagctagggccctgctaacacagctgcggtagagcagctgtgttagcagggccctagctccccaaaaccaaaa

SEQ ID NO: 52 - DNA, 52 bp, Artificial Sequence
  Synthetic - dR52
ggtgtctgtgccatcactcccatctcttccggcacagacaccaaaaccaaaa

SEQ ID NO: 53 - DNA, 52 bp, Artificial Sequence
  Synthetic - dR53
tcttccataaaaggtgtctgtgccggtagaggcacagacaccaaaaccaaaa

SEQ ID NO: 54 - DNA, 52 bp, Artificial Sequence
  Synthetic - dR54
ggtcaagaggccgcaccccccgcccaccacggcctcttgaccaaaaccaaaa

SEQ ID NO: 55 - DNA, 64 bp, Artificial Sequence
  Synthetic - dR55
ggtgtctgtgccatcactcccatctctccacccaatcacataggcacagacaccaaaaccaaaa

SEQ ID NO: 56 - DNA, 52 bp, Artificial Sequence
  Synthetic - dR56
ggtgtctgtgcctcttcccccatctctccaggcacagacaccaaaaccaaaa

SEQ ID NO: 57 - DNA, 52 bp, Artificial Sequence
  Synthetic - dR57
ggtgtctgtgccatcacttcttcctctccaggcacagacaccaaaaccaaaa

SEQ ID NO: 58 - DNA, 52 bp, Artificial Sequence
  Synthetic - dR58
ggtcaagaggccacatcctcagcaatcctcggcctcttgaccaaaaccaaaa

SEQ ID NO: 59 - DNA, 46 bp, Artificial Sequence
  Synthetic - dR59
gatcccggtgtctgtgccggtagaggcacagacaccaaaaccaaaa

SEQ ID NO: 60 - DNA, 58 bp, Artificial Sequence
  Synthetic - dR60
ggtgtctgtgccatcactcccatctctccatcctcaggcacagacaccaaaaccaaaa

SEQ ID NO: 61 - DNA, 52 bp, Artificial Sequence
  Synthetic - dR61
caaaaaaaaaaaggtgtctgtgccggtagaggcacagacaccaaaaccaaaa

SEQ ID NO: 62 - DNA, 40 bp, Artificial Sequence
  Synthetic - dR62
gtatttatattaggtagataatataaatacaaaaccaaaa

SEQ ID NO: 63 - DNA, 52 bp, Artificial Sequence
  Synthetic - dR63
tcttccataaaaggttcttccgcgtcttcccgcggaagaaccaaaaccaaaa

SEQ ID NO: 64 - DNA, 68 bp, Artificial Sequence
  Synthetic - dR64
cgcggagaccactcggggtcaacctaaatccaatcccggaccccgagtggtctccgcgaaaaccaaaa

SEQ ID NO: 65 - DNA, 52 bp, Artificial Sequence
  Synthetic - dR65
ggtcaagaggccaaatcatcaaaaattctgggcctcttgaccaaaaccaaaa

SEQ ID NO: 66 - DNA, 52 bp, Artificial Sequence
  Synthetic - dR66
ggtgtctgtgccatcacttcttcctcttccggcacagacaccaaaaccaaaa

SEQ ID NO: 67 - DNA, 52 bp, Artificial Sequence
  Synthetic - dR67
ggtgtctgtgcctcttcccccatctcttccggcacagacaccaaaaccaaaa

SEQ ID NO: 68 - DNA, 40 bp, Artificial Sequence
  Synthetic - dR68
catatattatacagctcagtataatatatgaaaaccaaaa

SEQ ID NO: 69 - DNA, 10 bp, Artificial Sequence
  Synthetic - dR69
aaaaccaaaa

SEQ ID NO: 70 - DNA, 58 bp, Artificial Sequence
  Synthetic - dR70
caaaaaaaaaaaaaaaaacggaccacctgccagacagcaggtggtccgaaaaccaaaa

SEQ ID NO: 71 - DNA, 64 bp, Artificial Sequence
  Synthetic - dR71
ggtcaagaggccacatcctcagcaatcctccatccctcaaagggcctcttgaccaaaaccaaaa

SEQ ID NO: 72 - DNA, 40 bp, Artificial Sequence
  Synthetic - dR72
aatatattataaggtagattataatatattaaaaccaaaa

SEQ ID NO: 73 - DNA, 40 bp, Artificial Sequence
  Synthetic - dR73
ttatttatattaggtagataatataaataaaaaaccaaaa

SEQ ID NO: 74 - DNA, 52 bp, Artificial Sequence
  Synthetic - dR74
ggtgtctgtgcctcttcctcttcctctccaggcacagacaccaaaaccaaaa

SEQ ID NO: 75 - DNA, 68 bp, Artificial Sequence
  Synthetic - dR75
cattaacatatattctgttcaacctaaatccaatcccggaacagaatatatgttaatgaaaaccaaaa

SEQ ID NO: 76 - DNA, 76 bp, Artificial Sequence
  Synthetic - dR76
gctcctgcaaatttatggggtcaagaggccggtagaggcctcttgaccccataaatttgcaggagcaaaaccaaaa

SEQ ID NO: 77 - DNA, 58 bp, Artificial Sequence
  Synthetic - dR77
caaaaaaaaaaaaaaaaaggtgtctgtgccggtagaggcacagacaccaaaaccaaaa

SEQ ID NO: 78 - DNA, 40 bp, Artificial Sequence
  Synthetic - dR78
gatatattataaagctcagtataatatatcaaaaccaaaa

SEQ ID NO: 79 - DNA, 58 bp, Artificial Sequence
  Synthetic - dR79
ggtcaagaggccacatcctcagcaatcctccatcccggcctcttgaccaaaaccaaaa

SEQ ID NO: 80 - DNA, 56 bp, Artificial Sequence
  Synthetic - dR80
cattaacatatattctgttccagacagaacagaatatatgttaatgaaaaccaaaa

SEQ ID NO: 81 - DNA, 52 bp, Artificial Sequence
  Synthetic - dR81
ggtgtctgtgcctcttcctcttcctcttccggcacagacaccaaaaccaaaa

SEQ ID NO: 82 - DNA, 52 bp, Artificial Sequence
  Synthetic - dR82
tcttcctcttccggttcttccgcgtcttcccgcggaagaaccaaaaccaaaa

SEQ ID NO: 83 - DNA, 21 bp, Artificial Sequence
  Synthetic - primer for 16S rRNA amplification
gaatgccacggtgaatacgtt

SEQ ID NO: 84 - DNA, 20 bp, Artificial Sequence
  Synthetic - primer for 16S rRNA amplification
cacaaagtggtaagcgccct

SEQ ID NO: 85 - DNA, 21 bp, Artificial Sequence
  Synthetic - primer for GFP amplification
cagtggagagggtgaaggtga

SEQ ID NO: 86 - DNA, 22 bp, Artificial Sequence
  Synthetic - primer for GFP amplification
cctgtacataaccttcgggcat

SEQ ID NO: 87 - DNA, 11 bp, Artificial Sequence
  Synthetic - strong RBS BBa_B0034
aaagaggagaa

SEQ ID NO: 88 - DNA, 10 bp, Artificial Sequence
  Synthetic - insulator sequence used in the dtRNAs
aaaaccaaaa

SEQ ID NO: 89 - DNA, 64 bp, Artificial Sequence
  Synthetic - common 3′ end sequence
tactagagaaagaggagaaatactagatgcgtaaaggagaagaacttttcactggagttgtccc

(19) United States
(12) Patent Application Publication
(10) Pub. No.: US 2023/0089497 A1
(43) Pub. Date: Mar. 23, 2023

(54) RATIONAL DESIGN OF UPSTREAM ENHANCEMENT RNA FOR CIRCUIT DYNAMICS REGULATION AND VIRAL DIAGNOSTICS OPTIMIZATION

(71) Applicant: Arizona Board of Regents on behalf of Arizona State University, Scottsdale, AZ (US)

(72) Inventors: Qi ZHANG, Tempe, AZ (US); Xiao WANG, Chandler, AZ (US); Alexander GREEN, Chestnut Hill, MA (US); Duo MA, Tempe, AZ (US); Fuqing WU, Tempe, AZ (US); Kylie STANDAGE-BEIER, Phoenix, AZ (US); Xingwen CHEN, Tempe, AZ (US)

(21) Appl. No.: 17/909,345

(22) PCT Filed: Mar. 3, 2021
(86) PCT No.: PCT/US2021/020698

§ 371 Date: Sep. 2, 2022

Related U.S. Application Data
(60) Provisional application No. 62/984,622, filed on Mar. 3, 2020.

(51) Int. Cl. C12N 15/67

(52) U.S. Cl. CPC C12N 15/67; C12N 2310/20

(57) ABSTRACT

The present invention provides a new class of RNA modules, referred to as degradation tuning RNAs (dtRNAs), which form stabilizing secondary structures. Also provided are methods of using dtRNAs to modulate the stability of RNAs. DNA constructs including a promoter that is operably connected to a sequence encoding the dtRNA are also provided.

### CROSS-REFERENCE TO RELATED APPLICATIONS

This application claims priority to U.S. Provisional Application No. 62/984,622 filed on Mar. 3, 2020, the contents of which are incorporated by reference in their entireties.

### STATEMENT REGARDING FEDERALLY SPONSORED RESEARCH

This invention was made with government support under 1100309 awarded by the National Science Foundation and GM106081, GM131405, GM126892, and AI136571 awarded by the National Institutes of Health. The government has certain rights in the invention.

### SEQUENCE LISTING

A Sequence Listing accompanies this application and is submitted as an ASCII text file of the sequence listing named “112624-01242_ST25.txt” which is 17.6 KB in size and was created on Mar. 1, 2021. The sequence listing is electronically submitted via EFS-Web with the application and is incorporated herein by reference in its entirety.

### BACKGROUND

Precise regulation of gene expression at the level of transcription or translation plays a pivotal role in establishing basic cell function, ensuring appropriate responses to environmental cues, and even robust therapeutics and diagnostics¹⁻⁶. Therefore, effective strategies are required to enable accurate and predictable control of the production and degradation of RNA and protein molecules⁷⁻⁹. In bacteria, such control has largely been achieved through engineering of the production of RNA (transcription) or protein (translation). Modulation of the −35 and −10 consensus elements has allowed for engineering of synthetic promoter libraries with a broad range of transcription efficiencies¹⁰⁻¹². This mechanism-driven methodology has also been applied to develop tools to manipulate translation, where RNAs featuring low folding energy coupled with high affinity Shine Dalgarno (SD) sequences to encourage efficient ribosome binding, thereby leading to accelerated translation rates^{13,14}. Libraries of ribosome binding sites (RBSs) with varying strengths have been developed to predict and tune protein yields¹⁴⁻¹⁶. Other attempts have been made to control the production of gene products by developing synthetic transcriptional terminators¹⁷⁻¹⁹, riboregulators²⁰⁻²³, thermosensors²⁴, ribozymes²⁵, CRISPR activation and interference systems²⁶⁻²⁹, switchable guide RNAs³⁰⁻³², engineering regions nearby open reading frames (ORFs)³³⁻³⁷, and through optimization of codon usage^{38,39}. However, there are significant limitations to these approaches. For example, methods such as engineering strong promoters require over production of genetic materials and thus introduce metabolic burden to the host cells, while others such as riboregulator or thermosensor are translation-dependent and therefore unable to manipulate RNA levels.

RNA molecules in prokaryotes are typically unstable, with half-lives on the minute timescale, which allows cells to rapidly adapt to changes in the environment^{40,41}. This rapid degradation is orchestrated by an ensemble of bacterial ribonucleases (RNases) that have been extensively studied^{42,43}. In E. coli, which lacks 5′→3′ exonucleases, the vast majority of RNA degradation processes combine the actions of endonucleases and 3′→5′ exonucleases. Specifically, the endonucleases RNase E or RNase III target the underlying RNA molecule for primary cleavage followed by complete degradation via 3′→5′ exonucleases⁴⁴. Previous studies have discovered several naturally occurring 5′ UTRs, termed RNA stabilizers, or rationally designed synthetic DNA cassettes that can increase RNA half-life by forming 5′ secondary structures⁴⁵⁻⁵⁰. These 5′ hairpin structures have been shown to be able to control heterologous mRNA half-life and have been used to regulate recombinant protein expression without introducing stress to host cells⁵⁰. However, most engineered 5′ stabilizing elements have been designed and tested on an ad-hoc basis. Thus, an understanding of the relationship between stabilizer structural features and mRNA half-life has remained elusive.

Accordingly, there remains a need in the art for improved, versatile methods of modifying gene expression in a cell.

### SUMMARY

In a first aspect, the present invention provides degradation tuning RNAs (dtRNAs). The dtRNAs comprise the following components, ordered from 5′ to 3′: (a) a leader sequence comprising zero to six nucleotides, (b) a first stem-forming region, (c) a loop-forming region comprising at least three nucleotides, (d) a second stem-forming region, and (e) an insulator sequence comprising at least five nucleotides. The first stem-forming region and the second stem-forming region of the dtRNAs form a stem that is at three nucleotides in length.

In a second aspect, the present invention provides methods of modulating the stability of an RNA. The methods comprise: (a) forming a dtRNA described herein; and (b) inserting the dtRNA into the RNA in a position that is 5′ to the functional portion of the RNA. In some embodiments, the methods increases the stability of the RNA. In other embodiments, the methods decrease the stability of the RNA.

In a third aspect, the present invention provides DNA constructs comprising a promoter that is operably connected to a sequence encoding a dtRNA described herein and a multi-cloning site or a functional RNA.

### BRIEF DESCRIPTION OF THE DRAWINGS

FIG. 1 shows modulation of RNA stability by native ompA stabilizer variants. a, Schematic showing the stabilizer protection mechanism and the plasmid constructed for fluorescence measurements. Engineered stabilizer variants are inserted between a constitutive promoter and the RBS to regulate GFP expression. Engineered stabilizer variants can form a hairpin structure (blue) to block RNase access. The structure depicted by a red dashed line indicates the small hairpin structure design nearby the RBS of WT_I, Hp1_I and Hp2_I. For the plasmid map, the gray arrow represents the constitutive promoter; the blue rectangle represents the RNA stabilizer; the orange oval represents the RBS; the green box represents GFP gene; the gray T represents the transcriptional terminator. b, Plate reader measurements shows that GFP fluorescence is affected by engineered stabilizer variants. The designs adopt the whole (WT) or part (Hp1 and Hp2) of the native ompA stabilizer and exhibit GFP fluorescence enhancement (blue). Low GFP expression is observed for circuits WT_I, Hp1_I and Hp2_I with small hairpin structures nearby the RBS region (red bars). The gray bar represents the control circuit result (Ctrl). Error bars are the SD of four biological replicates. * p<0.05, ** p<0.01, *** p<0.001 by student's t test. c, Comparison between relative mRNA level and relative GFP fluorescence for circuit WT, Hp1 and Hp2. The result shows a strong correlation between these two factors (R²=0.8997).

FIG. 2 depicts the identification of functional structural features of synthetic dtRNAs. a, Schematic showing the workflow for the present study. b-d, Correlations between each structural feature and the relative GFP expression. For all designs, 3′ insulation is achieved by insertion of ten single-stranded nucleotides downstream of the hairpin structure to minimize interference with the downstream RBS. b, Correlation between dtRNA stem GC content (0% to 100%) and the relative GFP fluorescence, and this result was fitted using smoothing spline (solid curve); c, Correlation between dtRNA stem length (3 bp to 30 bp) and the relative GFP fluorescence, and this result was fitted using smoothing spline (solid curve); d, Correlation between dtRNA loop size (3 nt to 30 nt) and the relative GFP fluorescence, this result was linear fitted (solid line, R²=0.806). The insets color-code the characterized structural features of dtRNA, and the green arrow represents GFP mRNA. The dashed line represents the control fluorescence level. Error bars are the SD of six biological replicates. e, (Left) Relative GFP fluorescence of synthetic dtRNA library. Orange bars represent designs with over 4-fold fluorescence enhancement; green bars represent designs with 2 to 4-fold enhancement; blue bars represent designs with 1-fold to 2-fold enhancement; gray bars represent designs with fluorescence lower than the control (c). Error bars are the SD of six biological replicates, with each data point represented by one black dot. Asterisks represent the dtRNAs used for in vitro measurement. Inset: Growth curve measurement results showing the OD 600 values for dR1, dR42, dR56 and control over 20 hours. Error bars are the SD of three biological replicates. (Right) Summary of GFP fold difference across dtRNA structures with the least and the most stable sequences, engineered stabilizer variant Hp1 (FIG. 1b) and the control. Over 40-fold dynamic range is achieved through optimization of functional structural features of the dtRNAs.

FIG. 3 shows the use of dtRNAs to modulate gene circuit dynamics and noncoding RNA levels in synthetic gene circuits. a, Schematic showing the construction of the LuxR/Luxl quorum sensing gene circuit where a constitutive promoter (gray arrow) triggers the expression of LuxR gene (purple rectangle). After being expressed, the LuxR protein dimerizes with 3OC6HSL (orange dots) and interacts with the pLux promoter to activate GFP gene expression (green rectangle). The blue rectangle represents the location of dtRNA insertion (dR1 and dR6). b, Dose-response measurement results induced by various 3OC6HSL concentrations. Error bars are the SD of four biological replicates. c, Hysteresis experiment results for the synthetic positive feedback loop (The circuit detail can be found in FIG. 10a). Various concentrations of 3OC6HSL are applied to induce each circuit. The purple lines indicate the result of initial OFF/ON experiment for the control circuit H Ctrl; The green lines indicate the result for circuit H_dR1; The blue lines indicate the result of initial OFF/ON experiment for circuit H_dR82. The zoomed in hysteresis result of 0 to 2 nM (dashed line) 3OC6HSL concentration can be found in FIG. 10b. The data represents the mean±SD of three biological replicates. d, Two-parameter bifurcation analysis result. The red lines mark the bifurcation between the monostability and bistability. The bistable region becomes smaller when shifted to lower drug concentration with the increasing of dtRNA strength. Parameter a are estimated based on our qPCR result. e, Schematic showing CRISPRi regulation controlled by dtRNAs. Selected dtRNAs (dR1, dR6, dR15 and dR19) are integrated with sgRNA which can guide dCas9 to repress GFP expression. f, Steady state fluorescence measurement for each CRISPRi system. All redesigned sgRNAs exhibit even lower GFP level compared to the original sgRNA (sgRNA_WT). Compared to the original sgRNA (sgRNA_WT) regulated CRISPRi, GFP expression yields about 22% to 36% decreasing when sgRNA is regulated by the dtRNAs. sgRNA_NC represents the negative control result. The data represents the mean±SD of six biological replicates. ** p<0.01, *** p<0.001 by student's t test.

FIG. 4 shows in vitro regulation of gene expression and RNA aptamer production via synthetic dtRNAs. a, Schematic showing the in vitro gene expression measurements with synthetic dtRNAs (dR4, dR7, dR15 and dR19). b, GFP expression measurement over time regulated by dtRNAs without (top)/with (bottom) RNase inhibitor treatment. Colored circles represent the observed mean GFP fluorescence of each design; solid lines represent model fitting results for each design (shown below). GFP fluorescence is measured every 50 seconds. c, Model simulation of GFP accumulation rate regulated by dtRNAs without (top)/with (bottom) RNase inhibitor treatment. d, Bar chart result shows the stabilizing efficacy of each dtRNA. Stabilizing efficacy is defined as the ratio between steady state GFP without RNase inhibitor and with RNase inhibitor treatment. The resultant values are further normalized against the control value. e, RNA aptamer assay result showing Broccoli aptamer fluorescence regulated by dtRNAs (dR4, dR7, dR15 and dR19). Colored circles represent the observed aptamer fluorescence; solid lines represent model fitting results for each design (Supplementary information, provided below). Aptamer fluorescence is measured every 90 seconds.

FIG. 5 shows redesigned hybrid dtRNA/toehold switch sensors improve the performance of in vitro paper-based viral diagnostics. a, Schematic showing the structure of redesigned toehold switch sensors and their recognition of target RNAs. The synthetic dtRNA is integrated upstream of the sensor for stabilization. During viral RNA recognition, the target RNA with a sequence X is recognized by the complementary X* region in the toehold switch. Binding through the single-stranded toehold region enables unwinding of the sensor hairpin to expose the RBS and start codon AUG for translation initiation. The synthetic dtRNA maintains its stable structure and protects the whole sensor transcript during the reaction. b, Norovirus diagnostics results without (top) and with (bottom) RNase inhibitor treatment. Each curve represents the average OD value of five reaction replicates. The details of each diagnostic result are shown in FIG. 15. c-d, Photographs and their corresponding diagnostic results for each sensor after 1- or 1.5-hour reactions with/without RNase inhibitor treatment, respectively. + represents the addition of synthetic norovirus RNA to the sensor. − represents the negative control. The dashed line indicates the detection threshold for each device (ΔOD575=0.4). The data represents the mean±SD of at least four biological replicates.

FIG. 6 shows the structure of naturally occurring ompA stabilizer and GFP expression measurement for circuits under a strong transcriptional promoter. (a) Schematic showing the structure of naturally occurring ompA stabilizer, which comprises two hairpin structures, hairpin_1 and hairpin_2. Single-stranded nucleotide sequence one (ss1) is located between two hairpins and single-stranded nucleotide sequence two (ss2) lies downstream of hairpin_2. (b-c) GFP fluorescence measurement results for circuits transcription under a strong promoter. (b) Design WT, Hp1 and Hp2 exhibits comparable GFP fluorescence. (c) Each design with small structure formation nearby RBS region shows low GFP fluorescence levels. The data represents the mean±SD of four biological replicates. n.s. (not significant) p>0.05, * p<0.05, ** p<0.01 by student t test.

FIG. 7 shows fluorescence measurements of synthetic dtRNAs with RNase E cleavage sites engineered into different structural regions. (a) Fifteen synthetic dtRNAs are designed without (-) or with single/multiple RNase E cleavage sites (UCUUCC) engineered into different structural regions of the stable dtRNA. The regions are marked for single or multiple RNase E cleavage sites insertion (right). Fluorescence measurement result shows that insertion of cleavage sites have insignificant effects on RNA stability. (b) Fluorescence measurement for dtRNAs with multiple RNase E cleavage sites inserting into 18-nt loop region. The inset shows the location for RNase E cleavage sites insertion. (c) Characterize the effect of dtRNA 5′ spacing length on GFP expression. Five dtRNAs with 5′ spacing lengths from 1-nt to 18-nt are designed to measurement their effect on GFP expression. The inset shows the location of dtRNA 5′ spacing region (pink). (d) Fluorescence measurement of dtRNAs with RNase E cleavage sites engineered into 12-nt 5′ spacing region. The inset shows the position of RNase E cleavage site (yellow). Error bars are the SD of six biological replicates.

FIG. 8 shows that other factors, including bulge, loop GC content, downstream gene, promoter, and RBS, have insignificant effects on dtRNA function. (a) Relative GFP expression of circuits regulated by dtRNAs with or without the bulge introduced in stem region. A three-nucleotide bulge was designed into stem region of dR1 and dR4 to be dR11 and dR26. There is no significant fluorescence difference among all designs indicating the introduction of bugle has little effect on GFP fluorescence enhancement. (b) Fluorescence measurements for designs with the same stem feature but varying loop GC content. We maintained 18-nt loop size and designed structures with 83.3%, 50% and 17.6% loop GC content, respectively. The result indicates that loop GC content also has minor effect on GFP variations. (c) Relative mRFP fluorescence regulated by selected dtRNAs with varying stabilizing abilities. Colors of the bar represent the fold enhancement of each dtRNA on GFP reporter. (d) Comparison between relative mRFP fluorescence and relative GFP fluorescence of selected dtRNAs. The result exhibits high correlation (R²=0.8681) between the report gene expression suggesting dtRNA performance is transferable to the other genes with different sequence composition. (e) Commonality test for circuits with different promoters (Top). Two promoters are selected (Biobrick number: J23105 and J23109, Table 1) and engineered into the circuit with identical constructions. GFP fluorescence measurement result shows that dtRNAs are able to enhance GFP fluorescence by different promoters (Bottom). (f) Commonality test for circuits with different RBSs (Top). We further engineered circuits with different RBSs (Biobrick number: B0031 and B0032 (Table 1). GFP fluorescence measurement results show that synthetic dtRNAs can upregulate the GFP fluorescence with different RBSs (Bottom). Error bars of each figure are the SD of six biological replicates.

FIG. 9 shows qPCR measurements of selected dtRNAs with varying stabilizing efficiency and the prediction of additional designed dtRNAs. (a) RT-qPCR measurement of relative RNA levels for dtRNAs with diverse stabilizing efficiency. The result displays a strong correlation between relative RNA levels and relative GFP fluorescence (R²=0.9406). Error bars of relative mRNA level are the SD of three biological replicates. (b) Relative fluorescence comparison between predicted relative GFP and observed relative GFP of dtRNAs constructed followed by combined design rules (Table 3). The result demonstrates that dtRNA stabilizing efficiency can be semi-quantitatively predicted based on each design rule, N is the total number for 54 single measurements regulated by additional designed dtRNAs (R²=0.5005). (c) Fluorescence measurement of dtRNA design f (Table 3) without (left) or with (right) 18 nt 5′ spacing. Error bars are the SD of six biological replicates. (d) Scatter plot reveals that structure MFE is not significantly correlated with GFP fluorescence enhancement regulated by synthetic dtRNA library (R²=0.000068).

FIG. 10 shows hysteresis measurements for engineered positive feedback loop H_dR6 and H_dR82 regulated by dtRNA. (a) Schematic showing the construction of positive feedback loop, dtRNA is only inserted at 5′ upstream of the LuxR gene. All genetic components are sharing the same colors as showed in FIG. 3a. (b) The hysteresis result of FIG. 3c regulated by dR1 and dR82 induced by 0 to 2 nM 3OC6HSL concentration. This figure serves to zoom in on lower induction doses shown in FIG. 3c to better visualize low dosage dynamics. (c) Hysteresis results for synthetic positive feedback circuit regulated by dR6 and dR81. Various concentrations of 3OC6HSL are applied to induce the circuit. The purple solid and dash lines indicate the control initial on and initial off experiment results; The green solid and dash lines represent H_dR6 initial on and initial off experiment results. The blue solid and dash lines represent H_dR81 initial on and initial off experiment results. The top panel is the enlarged result induced by 0 to 2 nM 3OC6HSL concentration. The data represents the mean±SD of three biological replicates.

FIG. 11 shows in vitro regulation of gene expression via synthetic dtRNAs (dR4, dR7, dR15 and dR19). (a) GFP fluorescence measurement results of designs without RNase inhibitor treatment. (b) GFP fluorescence measurement results of designs with RNase inhibitor treatment. The gray curve represents the mean fluorescence for circuit without dtRNA regulation (Ctrl). The purple, blue, green, and orange curves represent the mean fluorescence for circuits regulated by selected dtRNAs. The shallow area of each curve represents the SD of three biological replicates. GFP fluorescence is measured every 50 seconds.

FIG. 12 shows a comparison of relative GFP fluorescence and the results of an in vitro aptamer fluorescence assay. (a) Relative GFP fluorescence comparison among circuits regulated by the same dtRNAs in vitro and in vivo. Error bars are the SD of three biological replicates for in vitro measurement and six biological replicates for in vivo measurement (b) Aptamer fluorescence measurement assay. Black dots represent the fluorescence of control Broccoli aptamer; Gray dots represent Broccoli aptamer regulated by dtRNAs. (c) Comparison between in vivo relative GFP fluorescence and relative aptamer fluorescence in cell-free expression system. The result shows that there is little correlation between relative GFP and aptamer fluorescence, indicating the mechanisms of dtRNA regulation are different in each expression system. However, dtRNAs with short stem-loop hairpins tend to exert stronger positive effect on aptamer fluorescence (green dots). (d) Aptamer fluorescence measurements with varying 5′ single-stranded length. The result shows that aptamer fluorescence significantly reduces with longer 5′ single-stranded region, further suggesting excessive 5′ single strand can destabilize RNA molecules and could be easily targeted and digested by RNases.

FIG. 13 shows data fitting results for fluorescence of aptamers regulated by dtRNAs. Blue dots are the experimental data; Red lines are the model fitting curves.

FIG. 14 shows the in vitro norovirus diagnostics 2-h result and the expression leakage of each toehold sensor. (a) Leaky expression of sensors Ori, dR19_1 dR19_4 and dR19_5 without RNase inhibitor treatment. Leaky expression indicates the false positive result that reporter expresses even without viral input. (b) Plate reader measurement shows 2-hour viral diagnostics result without RNase inhibitor treatment. “+” represents groups induced by synthetic norovirus RNA and “—” represents the negative control; The dash line indicates the detection threshold for each device (ΔOD575=0.4). The data represents the mean±SD of five biological replicates. (c) Plate reader measurement shows device dR19_2 and dR19_3 exhibit high expression leakage. The data represents the mean±SD of five biological replicates. (d) Expression leakage of sensors Ori, dR19_1 dR19_4 and dR19_5 with RNase inhibitor treatment.

FIG. 15 shows norovirus diagnostic results for sensor Ori, dR19_1, dR19_4 and dR19_5. The shadow area for each sensor represents the SD of at least four biological replicates.

### DETAILED DESCRIPTION

The present application is based on the inventors' development of a new class of RNA modules known as “degradation tuning RNAs” or “dtRNAs,” which are designed to form stabilizing (or destabilizing) secondary structures. As described in the paragraphs that follow and the Example, the inventors engineered a library of dtRNAs that can be inserted at the 5′ end of RNAs of interest to manipulate their stability. Based on in silico analyses, the dtRNA modules form secondary structures that impact RNA degradation without interfering with downstream RNA features, including ribosome binding site (RBS) context. As is described in the Examples, the inventors systematically characterized dtRNA structures and discovered that RNA stability is strongly correlated with several structural features, including stem length, GC content, loop size, 5′ leader sequence, and the presence of ribonuclease (RNase) cleavage sites. Manipulation of these features yielded a library of 82 dtRNAs that can be used to tune gene expression upwards by 5-fold or downwards by 8-fold, resulting in an overall dynamic range of 40-fold. The sequences of these dtRNAs are provided herein as SEQ ID NO:1-82.

This disclosure further provides methods of using dtRNAs to modulate the stability of RNAs. In the Examples, the inventors demonstrate that integration of these dtRNAs can be used to tune the dynamics of a positive feedback loop or to increase noncoding RNA levels for improved CRISPR interference. They also show that dtRNAs can be used to tune gene and RNA aptamer production in in vitro cell-free systems, and can be used to improve paper-based viral diagnostics via integration into toehold switch sensors. This disclosure, therefore, provides a variety of dtRNAs that offer non-leaky and robust transcriptional regulation.

### Compositions:

The present invention provides degradation tuning RNAs (dtRNAs) comprising or consisting essentially of the following components, ordered from 5′ to 3′: (a) a leader sequence comprising zero to six nucleotides, (b) a first stem-forming region, (c) a loop-forming region, (d) a second stem-forming region, and (e) an insulator sequence comprising at least five nucleotides. The first stem-forming region and the second stem-forming region form a stem that is at three nucleotides in length. In some embodiments, the dtRNA contains only one stem loop.

The terms “stem loop,” “hairpin,” and “hairpin structure” refer to a lollipop-shaped RNA secondary structure formed two regions of a nucleic acid molecule (which are usually complementary when read in opposite directions) base pair to form a double helix that ends in an unpaired loop. As used herein, the term “stem” refers to the double-stranded portion of a stem loop, and the term “loop” refers to the single-stranded, unpaired portion of a stem loop.

The dtRNAs of the present invention comprise five essential components. On the 5′ end, the dtRNAs comprise a leader sequence comprising zero to six nucleotides. As used herein, the term “leader sequence” refers to the single-stranded region upstream (5′) of the stem loop-forming region within a dtRNA. The inventors have determined that a leader sequence is sometimes required for dtRNA stability. For example, the inventors have determined that a short leader sequence (i.e., GGG) is required for transcription by T7 RNA polymerase. Preferably, the leader sequence is about three to six nucleotides in length.

The dtRNAs of the present invention comprise a first stem-forming region and a second stem-forming region that form a stem that is at least three nucleotides in length. As used herein, the term “stem-forming region” refers to the portion of the dtRNA that forms the stem, i.e., the fully or partially double-stranded portion of a stem loop, formed via complementary base pairing. In some cases, the first and second stem-forming regions are perfectly complementary, such that all of the nucleotides in these regions participate in complementary base pairing. In other cases, the first and second stem-forming regions are not perfectly complementary, such that the stem loop comprises bulges, mismatches, and/or inner loops.

The dtRNAs of the present invention also comprise a loop-forming region comprising at least three nucleotides. As used herein, the term “loop forming region” refers to the portion of the dtRNA that forms the single-stranded loop of the stem-loop.

The dtRNAs of the present invention also comprise an insulator sequence comprising at least five nucleotides. As used herein, the term “insulator sequence” refers to a nucleotide sequence that has the ability to block the interaction of functional portions of a nucleic acid. In the present case, the insulator sequence is positioned downstream (3′) of the stem loop-forming region of the dtRNA to prevent interactions between the stem loop and any downstream portion of an RNA into which the dtRNA is inserted. Preferably, the insulator sequence is single stranded and does not form any unwanted hairpin structure that could affect the function of downstream RNA. Preferably, the insulator sequence is about 10 nucleotides in length. In certain embodiments, the insulator sequence is 5′-AAAACCAAAA-3′ (SEQ ID NO:88), a sequence that was designed by the inventors (i.e., using NUPACK) to interact minimally with surrounding sequences. However, the insulator sequence should be selected in view of the particular sequence context at hand. Ideally, the local RNA structure should be analyzed to prevent unwanted structure formation. Additionally, the insulator sequence should not contain functional sequences, such as transcriptional terminators or potential RNase cleavage sites that could negatively impact RNA function.

In some embodiments, the dtRNAs comprise one of the 82 synthetic dtRNAs that were tested by the inventors, which are disclosed herein as SEQ ID NO:1-82.

In another aspect, the present invention provides DNA constructs comprising the dtRNAs described herein. In some embodiments, the DNA constructs comprise a promoter that is operably connected to a sequence encoding the dtRNA and a protein. As used herein, the term “DNA construct” refers to an artificially constructed segment of DNA. In some cases, the DNA construct is a vector. The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors often comprise regulatory sequences, such as promoters and enhancers, which allow for expression of a polypeptide.

As used herein, the term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters that cause a gene to be expressed in most cell types at most times are commonly referred to as “constitutive promoters”. Promoters that allow the selective expression of a gene in most cell types are referred to as “inducible promoters”. Pol II or Pol III promoters may be utilized in the constructs provided herein and can be chosen by those of skill in the art for the particular purpose and RNA being generated by the construct.

As used herein, the term “operably linked” refers to a relationship between two nucleic acid sequences wherein the production or expression of one of the nucleic acid sequences is controlled by the other nucleic acid sequence. For instance, a promoter is operably linked to a nucleic acid sequence if the promoter is capable of affecting the expression of that sequence (i.e., the sequence is under the transcriptional control of the promoter).

The DNA constructs may encode any protein of interest. In the Examples, the inventors demonstrate that dtRNAs can also be applied to genes with very different sequence composition, i.e., GFP and mRFP, which have only 3% homology. Suitable proteins that may be encoded by the DNA constructs of the present invention include, for example, detectable reporter proteins (e.g., β-galactosidase, alkaline phosphatase, GFP, RFP, mCherry, luciferase), therapeutic proteins, and proteins of industrial interest. The DNA constructs may also encode RNA molecules such as iRNA, shRNAs, sgRNA for use in CRISPR/Cas gene editing or other functional RNA molecules encoded by a DNA and described more fully below. These RNAs may be under the control of a Pol III promoter.

### Methods:

The present invention provides methods of modulating the stability of an RNA. The methods comprise: (a) forming the dtRNA of claim 1 or 2; and (b) inserting the dtRNA into the RNA in a position that is 5′ to the functional portion of the RNA. When the modified RNA is transcribed, the dtRNA forms a hairpin structure that stabilizes and protects the transcribed RNA from RNase degradation.

In the Examples, the inventors demonstrate the ability of a dtRNA to stabilize or destabilize a RNA into which it is inserted. As used herein, the “stability” of a RNA refers to its half-life. In some cases, the addition of a dtRNA increases RNA stability by at least 2-fold. In some cases, RNA stability is increased at least 2-, 3-, 4-, 5-, 6-, 7-, 8-, or 9-fold or more, relative to a control lacking the dtRNA. In some cases, the addition of a dtRNA decreases RNA stability by at least 2-fold. In some cases, RNA stability is decreased at least 2-, 3-, 4-, 5-, 6-, 7-, 8-, 9-, 10-, 11-, 12-fold or more, relative to a control lacking the dtRNA.

In the present methods, the dtRNA may be formed using any suitable method, such as chemical synthesis or PCR mutagenesis. In some cases, the dtRNA will be provided as a complementary DNA (cDNA) that encodes the desired dtRNA sequence. The dtRNA sequence is inserted into a RNA of interest (or a DNA sequence encoding a RNA of interest) using standard molecular cloning techniques.

The methods of the present invention can be used to modulate the stability of any form of RNA. Suitable forms of RNA include, without limitation, messenger RNAs (mRNAs), transfer RNAs (tRNAs), ribosomal RNAs (rRNAs), microRNAs (miRNAs), siRNAs, piRNAs, snoRNAs, snRNAs, exRNAs, scaRNAs, long ncRNAs, and other synthetic RNAs (e.g., guide RNAs used in CRISPR-based systems). Thus, the term “functional portion” is used herein to refer generally to the portion of an RNA that either (1) encodes a protein, or (2) provides a non-coding RNA function (e.g., the portion of a miRNA that binds to a target sequence).

In the Examples, the inventors demonstrate that the ability of a dtRNA to stabilize or destabilize a RNA into which it is inserted is strongly correlated with several features of the dtRNA, which include the GC content of the stem-forming region, stem length, loop size, the length of the 5′ leader sequence, and the presence of RNase cleavage sites. Thus, the dtRNAs used in the methods of the present invention are characterized in terms of these features.

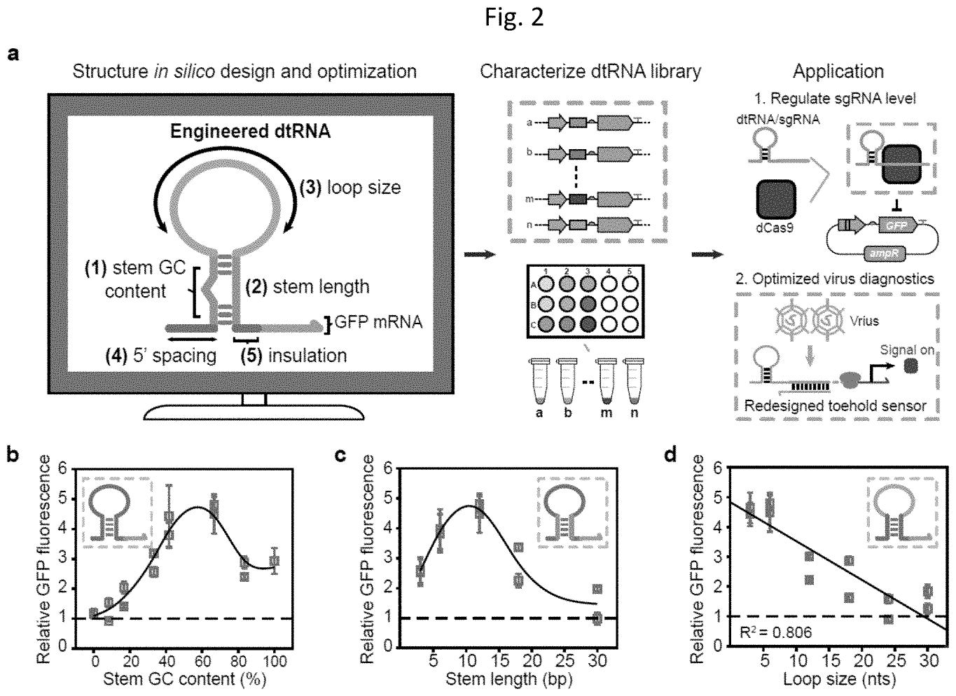

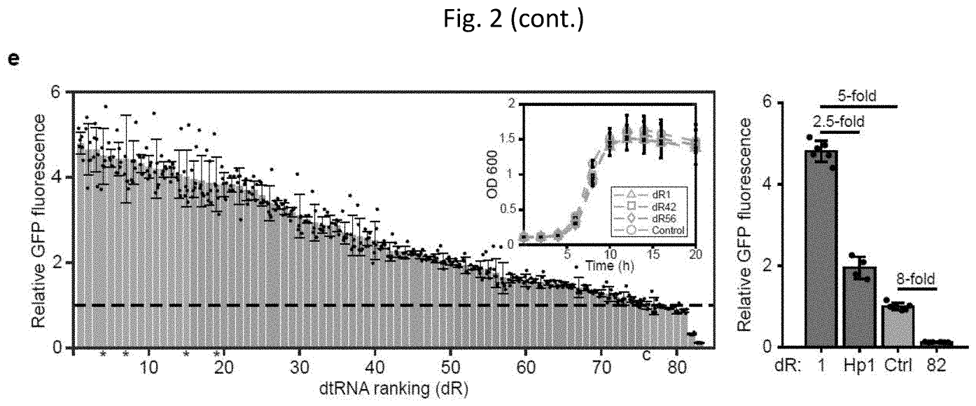

As used herein, the term “GC content” refers to the percentage of nitrogenous bases in a DNA or RNA molecule that are either guanine (G) or cytosine (C). As is demonstrated in FIG. 2B, dtRNAs with a moderate level of GC content in the stem-forming domain provide the greatest RNA stabilization. Thus, in some embodiments, the GC content of the dtRNA is between about 40% to about 80%. In certain embodiments, the GC content of the dtRNA is within a range that the inventors found to be maximally stabilizing, i.e., between about 41.6% and about 66.7%. In some embodiments the GC content of the stem region is maintained within these ranges, but the GC content of the remaining portions of the dtRNA may be distinct.

As shown in FIG. 2C, stem length also affects the stabilizing or destabilizing effect of a dtRNA. Stem lengths of 15 base pairs (bp) or more were associated with poor GFP fluorescence. However, GFP fluorescence was also diminished when the stem length was reduced to 3 bp. Thus, the stem length is preferably greater than 3 bp and fewer than 15 bp. In some embodiments, the stem is about 8 to about 15 base pairs in length. In certain embodiments, the stem is about 12 base pairs in length, which is the length that the inventors found to be maximally stabilizing. However, the exact length of the stem may be varied based on the particular sensitivity of the dtRNA and the application for which it is used. Notably the stem may include non-pairing bases, such that the stem comprises a bulge one or two nucleotides in length. The nucleotides making up the non-pairing portion of the stem do not count as part of the stem length because these bases are non-base pairing.

As shown in FIG. 2D, the loop size also affects the stabilizing or destabilizing effect of a dtRNA. It was empirically determined that a loop size of 3-nt, 4-nt, 5-nt, or 6-nt was associated with the greatest enhancement of RNA stability. Thus, in some embodiments, the loop-forming region is between about three nucleotides and about six nucleotides in length. In some embodiments, the loop-forming region is about four nucleotides in length. Without wishing to be bound to any particular theory, increasing loop size may increase the possibility for RNase targeting, thereby weakening RNA stability.

The leader sequence of the dtRNAs also affects RNA stability. Long single-stranded regions make RNAs unstable because they are targets for digestion by RNases. In the Examples, the inventors determined that the leader sequence only began to destabilize the RNAs when it was at least 18 nucleotides in length. Thus, in some embodiments, the leader sequence is less than 18 nucleotides in length. In other embodiments, the leader sequence at least 18 nucleotides in length.

The addition of RNase cleavage sites is known to destabilize RNAs. For example, the inventors have demonstrated that the addition of the RNase E cleavage site UCUUCC to an unstable dtRNA loop decreases RNA stability. Thus, in some embodiments, the dtRNA comprises one or more RNase E cleavage sites. However, any RNase cleavage site may be used in the dtRNAs of the present invention to allow for tunability of expression, i.e. to allow for precise regulation or alteration of gene expression.

The methods of the present invention may be used to either increase or decrease the stability of a RNA. The overall effect of inserting a dtRNA into a RNA sequence will depend on the dtRNA's specific combination of features that affect RNA stability. The dtRNAs tested by the inventors were capable of tuning expression upwards by up to 5-fold or downwards by up to 8-fold. Thus, the stabilizing or destabilizing effect of dtRNAs is tunable, and is readily modulated by the manipulation of the features described herein.

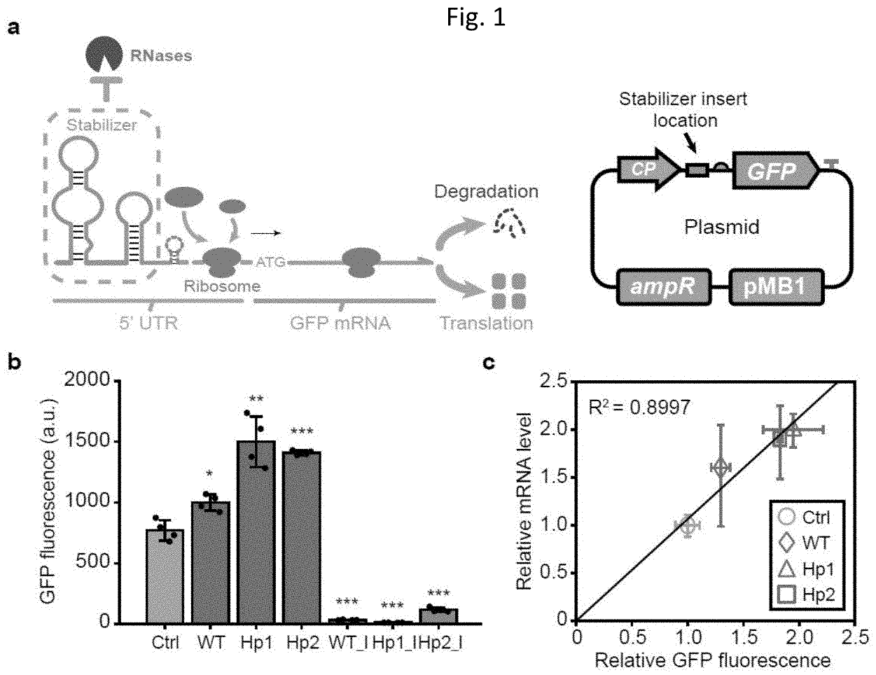

The methods of the present invention can be used to alter the stability of any form of RNA. In some embodiments, the methods are used to alter the stability of messenger RNA. The term “messenger RNA (mRNA)” refers to an RNA that encodes at least one protein. In these embodiments, the dtRNA is inserted between the transcription start site and the ribosome binding site of a DNA molecule encoding the mRNA. The term “transcription start site (TSS)” refers to the position in a DNA molecule from which transcription begins. The term “ribosome binding site (RBS)” refers to a sequence of nucleotides found upstream of the start codon of an mRNA transcript that is responsible for the recruitment of a ribosome during the initiation of translation. Positioning the dtRNA between the TSS and TBS ensures that the dtRNA is transcribed and that the insulator sequence at the 3′ end of the dtRNA will prevent the dtRNA stem loop from interfering with translation. For example, as is illustrated in FIG. 1A, the dtRNA is inserted 3′ (downstream) of a constitutive promoter and 5′ (upstream) of a sequence that comprises a ribosome binding sequence (RBS) and the functional portion of the mRNA (in this case, the portion encoding GFP) In the Examples, the inventors demonstrate that, by increasing mRNA stability, insertion of a dtRNA into an mRNA can be used to increase protein expression. Thus, in some embodiments, insertion of the dtRNA increases the expression of a protein encoded by the mRNA.

In some embodiments, the methods are used to alter the stability of a noncoding RNA. The term “noncoding RNA (ncRNA)” refers to a RNA that is not translated into a protein. In these embodiments, the dtRNA is inserted on the 5′ end of the ncRNA, i.e., 5′ to the functional portion of the ncRNA.

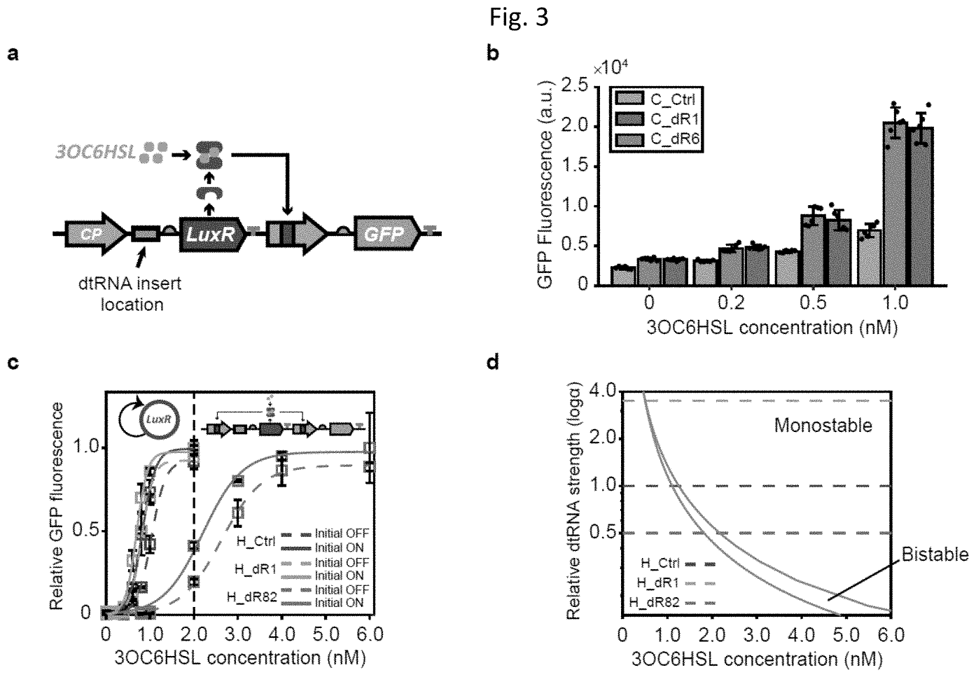

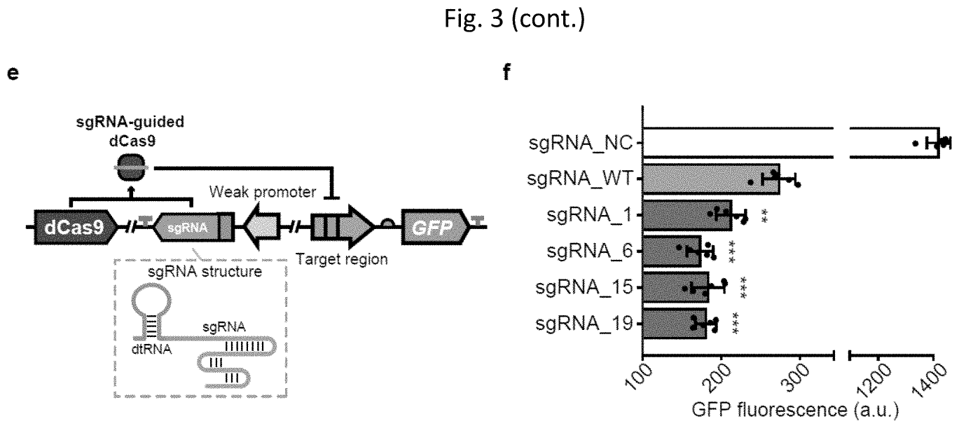

In some embodiments, the ncRNA is part of a CRISPR-based system. As used herein, the term “CRISPR-based system” refers to any system that utilizes CRISPR technology. Examples of CRISPR-based system include, without limitation, CRISPR-mediated genome editing, CRISPR-mediated epigenetic editing, CRISPR-mediated chromatin immunoprecipitation, CRISPR-mediated transcriptional activation, CRISPR-mediated transcriptional repression, CRISPR-mediating live imaging of DNA/RNA, and CRISPR libraries for screening. In some embodiments, a dtRNA is used to modulate the stability of a guide RNA used in a CRISPR-based system. As illustrated in FIG. 3E, including a dtRNA in the 5′ sequence of a small guide RNA (sgRNA) that targets a promoter driving the expression of GFP resulted in increased inhibition of GFP expression. Here, insertion of the dtRNA is believed to have increased the stability of the sgRNA, increasing the probability that it would target dCas9 to the target promoter sequence to inhibit the expression of GFP. As shown in FIG. 3F, fluorescence measurements show significantly lower GFP expression when dCas9 is guided by dtRNA-containing sgRNAs (about 22% to 36% decrease) relative to sgRNAs that were not modified to contain a dtRNA (“sgRNA_WT”). Thus, these data demonstrate that noncoding RNA levels can be regulated by dtRNAs.

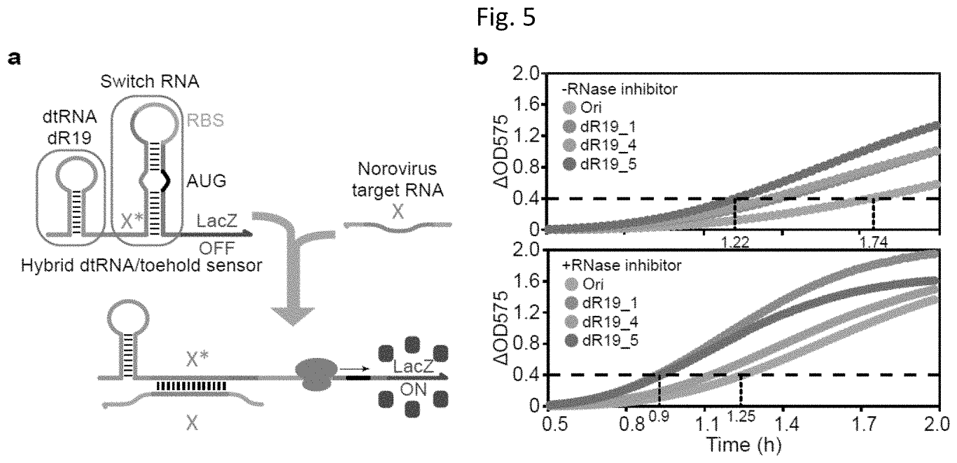

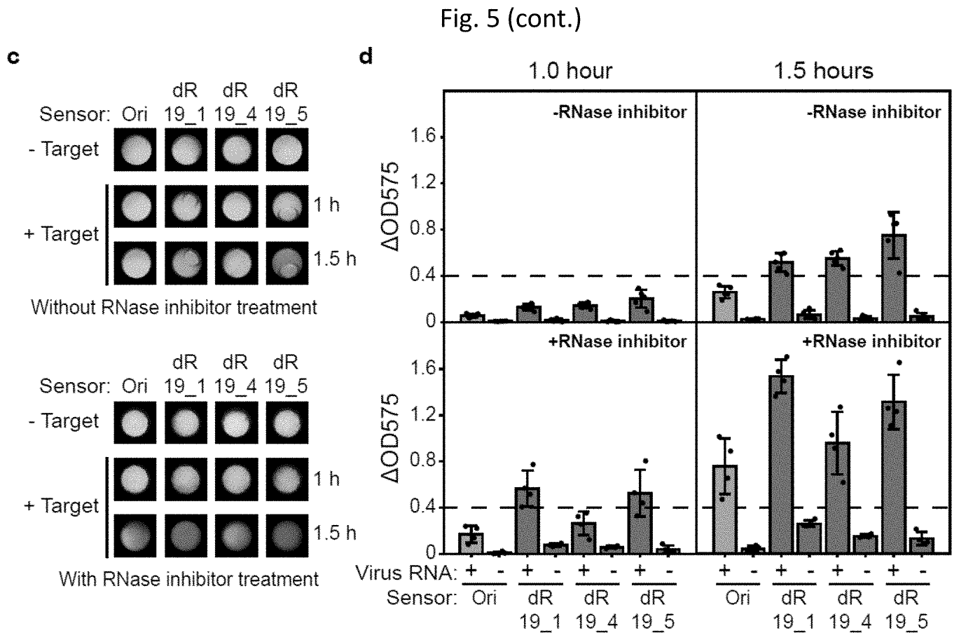

In some embodiments, the ncRNA comprises a toehold switch. As used herein, the term “toehold switch” refers to a class of RNAs that comprise a hairpin loop that unfolds upon binding to a cognate “trigger RNA” (i.e., an RNA comprising a region that is complementary to a portion of the toehold switch). Unfolding of the hairpin loops exposes a ribosome binding site (RBS) and permits translation of a downstream protein (Green et al., 2014, Cell 159:925-939). Thus, toehold switches are programmable RNA devices that are used to regulate translation. Generally, a toehold switch is designed to comprise a long 5′ single-stranded region that is complementary to the trigger RNA. Such long single-stranded regions make RNAs unstable, as they are targets for digestion by RNases. As is demonstrated in FIG. 5A, insertion of a dtRNA can be used to improve toehold switch stability.

In some embodiments, the dtRNAs are used to amplify detection signals in a diagnostic method or device. For example, in some cases, dtRNAs of this disclosure are added to RNAs that are used to detect the presence of a pathogen-associated nucleic acid in a sample. In some embodiments, the methods described herein are adapted for high-throughput or rapid detection, for example, in a clinical setting or in the field. When a dtRNA output is coupled to a reporter element, such as fluorescence emission or a color-change through enzymatic activity, the resulting synthetic molecule serves as a genetically encoded sensor for nucleic acid detection. In the Examples, the inventors demonstrate that adding dtRNAs to toehold switch sensors designed to detect norovirus-associated nucleic acids enhances the performance of a paper-based norovirus diagnostic assay. However, the methods of the present invention can be used to improve detection of any nucleic acid of interest. For example, other applications of the methods provided herein include, without limitation, detecting pathogens or environmental contaminants, profiling species in an environment (e.g., water, mosquito populations carrying mosquito-borne viruses); profiling species in an human or animal microbiome; food safety applications (e.g., detecting the presence of a pathogenic species, determining or confirming food source/origin such as type of animal or crop plant); obtaining patient expression profiles (e.g., detecting expression of a gene or panel of genes (e.g., biomarkers); wastewater monitoring applications (e.g., detecting the presence of pathogens in sewage for pathogen surveillance).

In the Examples, the inventors demonstrate that the inventors demonstrate that insertion of a dtRNA modulates the stability of an RNA both in vivo and in in vitro cell-free expression systems. Thus, in some embodiments, the RNA modulated by the methods of the present invention is expressed in a cell-free expression system. As used herein, the term “cell-free expression system” refers to a system in which protein is expressed in a crude extract rather than in a cell.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### Examples

The ability to tune RNA and gene expression dynamics is greatly needed for biotechnological applications and has motivated the development of an assortment of engineered libraries of components, including promoters, ribosome binding sites, and transcriptional terminators. RNA and protein levels are both strongly affected by transcript stability. Native RNA stabilizers or engineered 5′ stability hairpins have been utilized to regulate transcript half-life to control recombinant protein expression. However, these methods have been mostly ad-hoc and hence lack predictability and modularity. In the following Example, the inventors report a library of RNA modules called degradation tuning RNAs (dtRNAs) that can increase or decrease transcript stability in vivo and in vitro. dtRNAs enable modulation of transcript stability over a 40-fold dynamic range in Escherichia coli while having a minimal influence on translation initiation. They harness dtRNAs in mRNAs and noncoding RNAs to tune gene circuit dynamics and enhance CRISPR interference in vivo. Use of stabilizing dtRNAs in cell-free transcription-translation reactions also tunes gene and RNA aptamer production in vitro. Finally, they combine dtRNAs with toehold switch sensors to enhance the performance of paper-based norovirus diagnostics, illustrating the potential of synthetic dtRNAs for biotechnological applications.

Results:

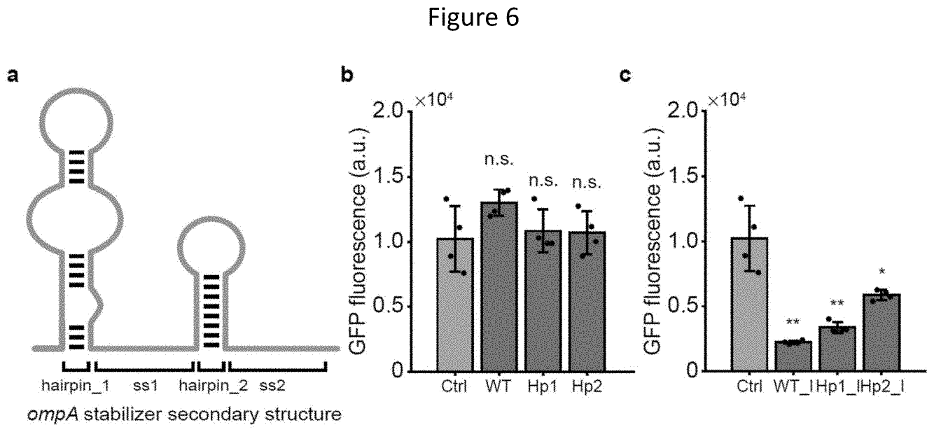

Modulation of RNA stability by variants of the native ompA stabilizer. Inspired by previous studies that naturally occurring stabilizers can be used to tune gene expression in synthetic gene circuits^{48,50}, we inserted the 5′ UTR sequence from the E. coli ompA transcript between the promoter and RBS region to tune downstream GFP expression^{33,45,47} (FIG. 1a, right). The RNA sequence of the stabilizer forms secondary structures to stabilize the mRNA following transcription (FIG. 1a, left). It can be seen in FIG. 1b that the wild-type (WT) stabilizer does indeed increase GFP levels moderately compared to a control (Ctrl) mRNA lacking the stabilizer sequence. Sequence analysis shows that the ompA stabilizer forms two hairpins (hairpin_1 and hairpin_2, blue structures in FIG. 1a) and two single-stranded sequences between the two hairpins (ss1) and downstream of hairpin_2 (ss2) (FIG. 6a). To investigate the contribution of these components to maintaining RNA stability, we designed and synthesized two variants of the ompA stabilizer: “Hp1” includes hairpin_1 and the first 7 nucleotides of ss1, and “Hp2” includes hairpin_2 and the first 7 nucleotides of ss2. Using a plate reader to measure GFP fluorescence after 16 hours of incubation, we first tested each cassette on a high-copy plasmid driven by a strong promoter but did not observe any significant fluorescence enhancements (FIG. 6b). To alleviate potential saturation of the transcription and degradation process, each cassette was then inserted into the plasmid driven by a weak promoter. Interestingly, both “Hp1” and “Hp2” displayed greater GFP expression than the WT ompA sequence, with design “Hp1” providing about a 2-fold increase in GFP over the control (FIG. 1b).

To explore the impact of extra secondary structures formed close to the RBS on GFP expression, another three stabilizer variants were designed and synthesized: “WT_I”, “Hp1_I” and “Hp2_I” which, compared to above designs, form eight extra base pairs with their downstream sequence to establish a short hairpin structure near RBS (red structure in FIG. 1a). These three designs showed weaker or no fluorescence (FIG. 1b and FIG. 6c), demonstrating that RNA secondary structure can interfere with translation when it is too close to the RBS, as expected from previous reports^{14,51}.

To rule out the possibility that the observed increase GFP fluorescence was due to enhanced translation rather than increased RNA stability, RT-qPCR experiments were carried out for Ctrl, WT, Hp1, and Hp2 to measure their RNA levels. FIG. 1c shows that RNA level variations can explain about 90% of the change of their corresponding GFP fluorescence (R²=0.8997), indicating that the observed fluorescence enhancements can be attributed primarily to increased RNA levels. These results demonstrated the viability of using artificial upstream 5′ UTR sequences to modulate RNA stability in our synthetic system. In addition, studying variants of naturally occurring RNA stabilizers helped reveal two general principles for their effective design: use of hairpin structures and establishing an appropriate distance between the hairpin and the RBS.

Identifying functional structural features of synthetic dtRNAs. Using these two general principles for stabilizers as a framework, we proceeded to design a library of synthetic dtRNAs with a range of structural features to systematically evaluate their influence on RNA stability. In silico analyses highlighted stem length, stem GC content, loop size, 5′ spacing sequence, and 3′ insulation as the primary candidate features to investigate as part of the library (FIG. 2a and design methods).

FIG. 2b displays quantitative characterization of the impacts of stem GC content on RNA stability. Theoretically, stems with high GC content are more thermodynamically stable and could lead to stronger enhancements of RNA stability. Fifteen dtRNAs with the same secondary structure (6-nt loop and 12-bp stem) but varying stem GC content were designed and tested (FIG. 2b). Fluorescence measurements show that structures with low GC content (less than 20%) nearly abolish the GFP expression enhancements, likely due to the unwinding of unstable AU rich hairpins removing their potential RNA-stabilizing effects. On the other hand, as the fraction of GC base pairs increases, GFP fluorescence increases concomitantly until it peaks at 66.7% GC content (8 out of 12 GC base pairs). With higher GC content, we observe diminished expression enhancement, presumably because RNA structures with GC-rich stem loops could act as transcriptional terminators, which stall RNA polymerases and cause the transcriptional complex to fall off and therefore lead to lower expression levels^{52,53}. This result quantifies the non-monotonic relationship between GC content and resulting RNA stability and also identifies that medium level (from 41.6% to 66.7% in our result) GC content is ideal for dtRNA structures to maximally enhance RNA stability.

To investigate the impact of stem length on RNA stability, another ten dtRNAs sharing the same loop sequence and optimal stem GC content but varying stem length were designed and tested (FIG. 2c). Fluorescence measurements show that structures with long stem lengths (30 bp) nearly eliminate RNA stability enhancement, possibly because even perfectly paired hairpins that are over 30 bp in length are likely to be targeted by RNase III to initiate the RNA degradation process⁴⁴. GFP fluorescence reaches its highest value for stem lengths of 12 bp. Further reductions in stem length lead to decreased hairpin stability and increased susceptibility to RNases as the stem is decreased down to 3 bp. These effects thus result in the non-monotonic relationship between stem length and the resulting RNA stability, where hairpins with 12-bp stems show the maximum RNA stability enhancement.

Finally, to identify the relationship between loop size and RNA stability, we designed and tested another set of twelve dtRNA structures containing optimal stem features but varying loop sizes. In theory, tetraloops, which are hairpin loops of 4 nt, endow an RNA structure with strong thermal stability and make them highly nuclease resistant⁵⁴. This effect is confirmed experimentally in FIG. 2d where structures with loop sizes of around 4 nt (3 nt and 6 nt in our result) display the highest RNA stability enhancement. GFP fluorescence levels decrease with enlarging loop size, likely because large loops increase the possibility for RNase targeting and thereby weaken RNA stability. Increasing loop sizes also increase the entropic cost associated with hairpin formation, making the hairpin less thermodynamically stable. These results demonstrate a monotonically decreasing relationship between loop size and RNA stability and establish that a loop size of around 4 nt (3 nt to 6 nt) is ideal for RNA stability enhancement.

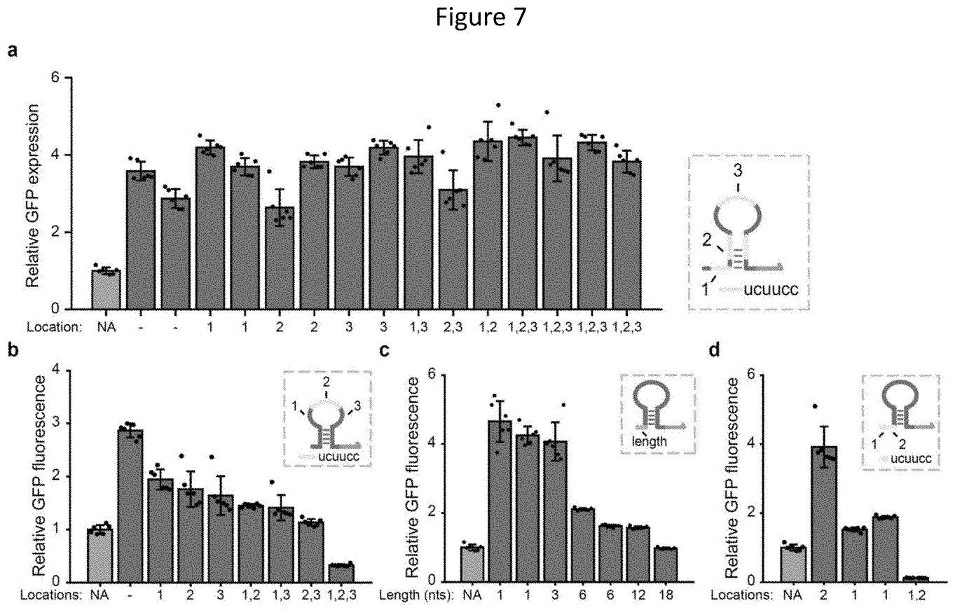

Having designed the necessary structural features to enhance RNA stability, we next explored incorporating motifs to decrease RNA stability. We first attempted to insert the previously reported RNase E cleavage site (UCUUCC, 6-nt) into dtRNA structures^{24,55}. No significant GFP fluorescence decrease was observed when cleavage sites were inserted into the stable hairpin (FIG. 7a). However, GFP fluorescence was significantly reduced when introducing three cleavage sites into the relatively unstable large loop hairpin structure, demonstrating that stabilizers with relatively “open” structures are readily targeted by RNases (FIG. 7b). We next interrogated the impact of a 5′ spacing sequence on RNA stability reduction. Unlike a previous report that found that as little as a 5-nt single-stranded region at the 5′ end of the RNA could completely abolish the stabilizer function⁴⁶, we observed RNA stability enhancement for structure with 12-nt single-stranded sequence. Indeed, the stabilizing effect is completely abolished only when the 5′ single-stranded region reaches 18 nt in length (FIG. 7c). We combined these two features by inserting RNase E cleavage site into the 5′ spacing sequence to test if RNA stability can be further decreased. As expected, GFP fluorescence decreased when the cleavage site is inserted 6 nt away from the hairpin structure, and the fluorescence level is even further downregulated by about 8-fold below the control when two RNase E cleavage sites are inserted (FIG. 7d).

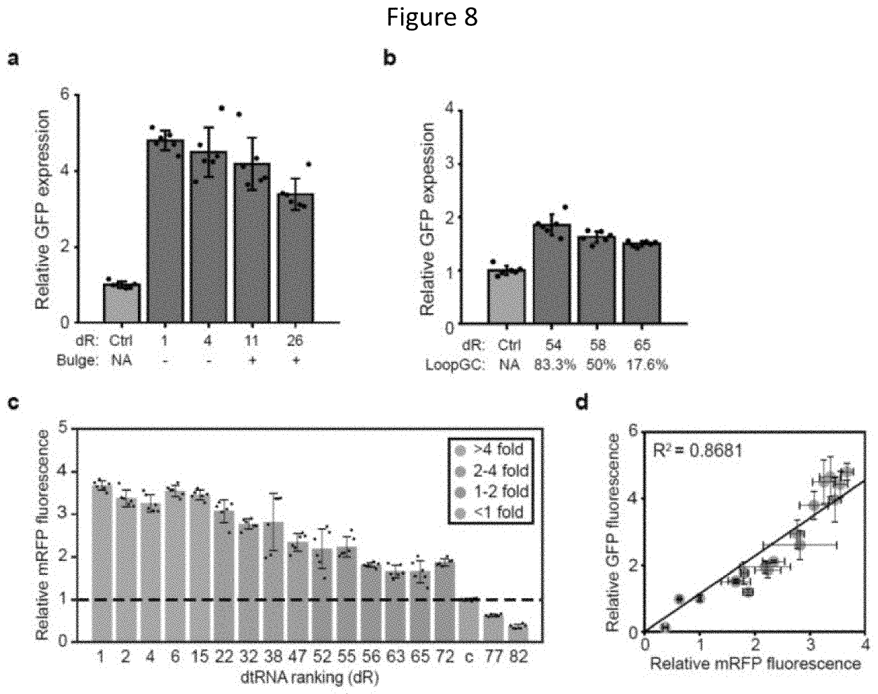

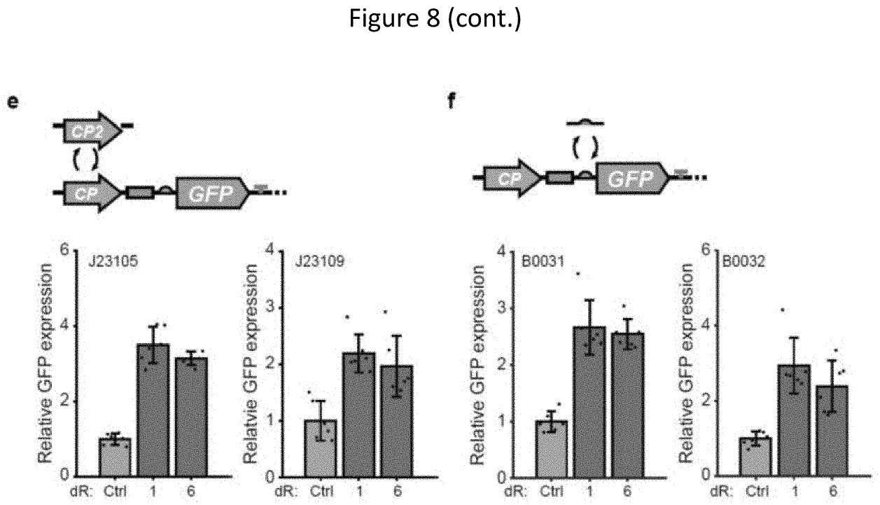

We also investigated other features such as the presence of bulges within the stem and loop GC content and found that they have insignificant effects on RNA stability (FIGS. 8a, b). To investigate if dtRNAs can also be applied to genes with very different sequence composition, we select dtRNAs with varying stabilizing capabilities to regulate mRFP expression. Sequence comparison analysis shows only 3% homology between GFP and mRFP gene, indicating that the mRFP reporter has a very different sequence composition compared to GFP. Following the same circuit construction scheme, we inserted each dtRNA upstream of mRFP to measure their effect on reporter expression. Fluorescence measurements show that dtRNAs with higher stability enhancements in the GFP library also displayed higher relative mRFP fluorescence (FIG. 8c). We further compared the mRFP performance of the selected dtRNAs to their GFP performance. The results also exhibit high correlation (R²=0.8681), suggesting dtRNA performance is transferable to other genes with different sequence compositions (FIG. 8d). To further verify that RNA stability enhancement is independent of genetic context, two dtRNA variants displaying high stability enhancements were measured with different promoters and RBSs (FIGS. 8e, f). These studies also showed that the dtRNAs retained their RNA stabilizing effect despite the change in genetic context.

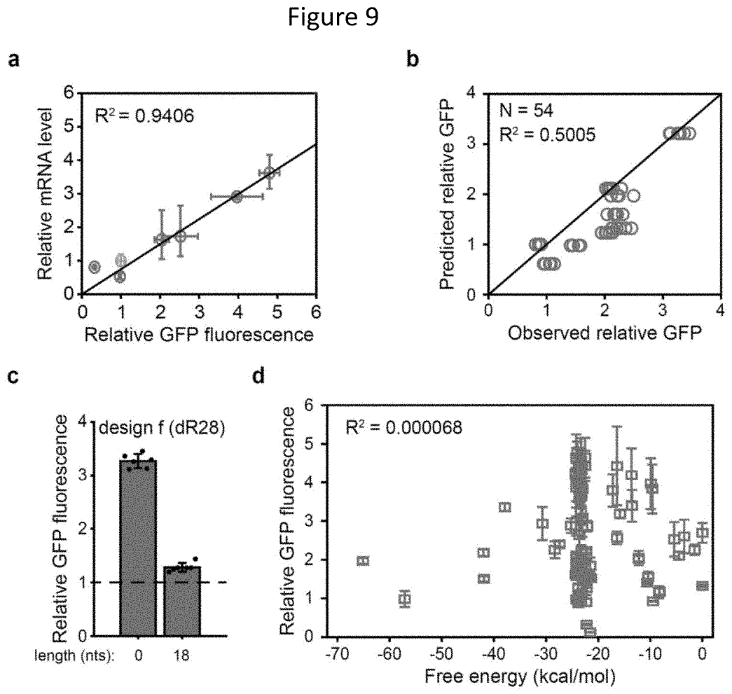

To confirm that the observed gene expression tuning could be attributed to RNA levels, RT-qPCR experiments were performed to measure RNA levels for selected dtRNAs with a range of GFP fluorescence enhancement levels. The results show a strong correlation between relative RNA level and relative GFP fluorescence (R²=0.9406), indicating that GFP fluorescence variation is mainly due to the change in RNA levels (FIG. 9a). To determine if dtRNA stabilizing capacity could be predicted, we designed additional dtRNAs with a range of structural features and calculated their predicted relative GFP levels based on the relationships between structure and stability shown in FIG. 2b-d (see Table 3 for design information). Fluorescence measurements show a strong correlation between the predicted and observed GFP levels (R²=0.5005, FIG. 9b). We also increased the 5′ spacing length of the dtRNA with the highest predicted stabilizing capacity among the new designs (design f, Table 3). Similar to our previous result, we found that the stabilizing effect is nearly abolished with long 5′ single-stranded regions (FIG. 9c). These results demonstrate that dtRNA stability enhancement ability can be semi-quantitatively predicted based on a few design rules.

Table 3. Information on additional dtRNAs constructed (a-i) followed by combined design rules and their predicted relative GFP. We defined three factors α, β and γ, which are calculated through the fitted GFP fluorescence of each feature (stem GC content (α), stem length (β) and loop size (γ)) based on FIG. 2b-d fitted curve divided by the maximum relative GFP fluorescence of each feature calculated via the fitted curve. In particular, we used smoothing spline to fit the GFP fluorescence regulated by dtRNAs with different stem GC content (R²=0.9381) and stem length (R²=9131) and linear fit the result influenced by varying loop size (R²=0.806):

$$α = \frac{fitted GFP ( stem GC content )}{Max GFP ( stem GC content )} \\ β = \frac{fitted GFP ( stem length )}{Max GFP ( stem length )} \\ γ = \frac{fitted GFP ( loop size )}{Max GFP ( loop size )}$$

We further averaged Max GFP from all three features as Max GFP (average) and the predicted relative GFP can be calculated by the equation (we assume that each feature impact the GFP fluorescence independently):

Predicted relative GFP=α·β·γ·Max GFP(average)

| | | | | Predicted | Predicted | Predicted | |
|---|---|---|---|---|---|---|---|
| dtRNA | Stem GC | Stem | Loop | factor | factor | factor | Predicted |
| Design | content | length | size | stem GC | stem length | loop size | relative |
| index | (%) | (bp) | (nt) | (α) | (β) | (γ) | GFP |
| a | 25 | 4 | 6 | 0.4864 | 0.6437 | 0.9118 | 1.3222 |
| (dR43) | | | | | | | |
| b | 25 | 20 | 6 | 0.4864 | 0.4869 | 0.9118 | 1.0001 |
| (dR80) | | | | | | | |
| c | 25 | 12 | 18 | 0.4864 | 0.9783 | 0.5592 | 1.2322 |
| (dR50) | | | | | | | |
| d | 25 | 20 | 18 | 0.4864 | 0.4869 | 0.5592 | 0.6133 |
| (dR75) | | | | | | | |
| e | 75 | 4 | 6 | 0.7777 | 0.6437 | 0.9118 | 2.114 |
| (dR48) | | | | | | | |
| f | 75 | 12 | 6 | 0.7777 | 0.9783 | 0.9118 | 3.2127 |
| (dR28) | | | | | | | |
| g | 75 | 20 | 6 | 0.7777 | 0.4869 | 0.9118 | 1.5991 |
| (dR46) | | | | | | | |
| h | 75 | 12 | 18 | 0.7777 | 0.9783 | 0.5592 | 1.9702 |
| (dR44) | | | | | | | |
| i | 75 | 20 | 18 | 0.7777 | 0.4869 | 0.5592 | 0.9806 |
| (dR64) | | | | | | | |

In all, we systematically designed and tested a library of 82 synthetic dtRNAs and identified the functional structural features affecting RNA stability. Each dtRNA shares a single hairpin structure with an insulator sequence at the 3′ end to prevent interference between the stability hairpin and RBS region. By tuning combinations of structural features, dtRNAs enable quantitative control over gene expression with a wide dynamic range of 40-fold from the least to the most stable sequences (FIGS. 2e, dtRNA stability ranked 1 through 82, denoted dR 1-82). We also note that no significant correlation between the dtRNA minimum free energy (MFE) and GFP fluorescence was detected (FIG. 9d), indicating that a combination of RNA sequence and structural features, rather than RNA folding alone, define transcript stability.

Modulation of gene circuit dynamics and noncoding RNA levels. As an initial test of the utility of dtRNAs, we selected two dtRNAs with the top GFP enhancement performance (dR1 and dR6) to incorporate into a LuxR/Luxl quorum sensing (QS) regulatory circuit and measure their impact on downstream GFP expression. It can be seen in FIG. 3a that synthetic dtRNAs are only inserted in the 5′ region upstream of the LuxR sequence to regulate LuxR expression (circuit C_dR1 and C_dR6). GFP fluorescence was measured to quantify the dose-response readout of each circuit. It can be seen in FIG. 3b that as the 3OC6HSL induction increases, GFP fluorescence increases by C_dR1 and C_dR6 become more pronounced when compared against the circuit without dtRNA regulation (C_Ctrl), suggesting synthetic dtRNAs are capable of stabilizing LuxR mRNA and thereby enhancing downstream GFP fluorescence in synthetic gene circuit (FIG. 3b). Such stability enhancement is amplified in high induction cases because of increased transcript abundance.

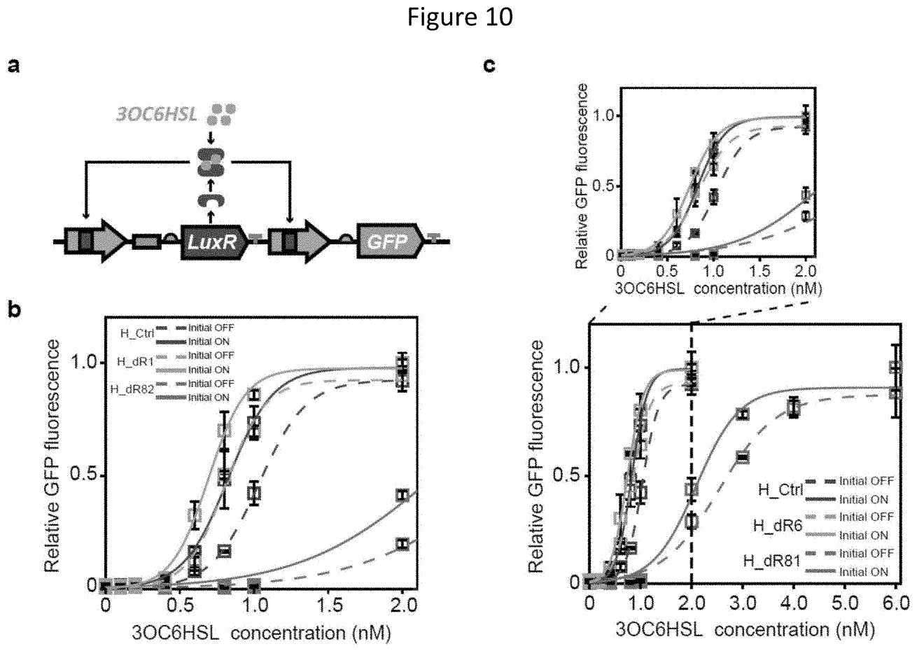

To explore this impact on nonlinear gene circuit dynamics, synthetic dtRNAs were inserted into a LuxR/Luxl QS-based positive feedback loop to tune the bistability of each circuit^{56,57}. The constitutive promoter in circuits C_dR1 and C_dR6 was replaced with a pLux promoter such that LuxR gene can activate itself to form a positive feedback topology (circuit H_dR1 and H_dR6) (FIG. 10a). Two weak dtRNAs (dR81 and dR82) were also inserted to tune the behavior of positive feedback circuit (H_dR81 and H_dR82). We measured the robustness of history-dependent response (hysteresis), the hallmark of positive feedback topology, to determine the dynamics of each circuit^{58,59}. A small bistable region is first observed for circuit H Ctrl without dtRNA regulation (FIG. 3c, purple lines). The bistable regions of circuit H_dR1 and H_dR6 regulated by dtRNA structures shifted to lower 3OC6HSL concentration because of increased LuxR transcript stability and in turn increased LuxR protein abundance, which make it easier for the system to switch to the ON state (FIG. 3c and FIG. 10b, c, green lines). We also observed enlarged bistable regions for circuits regulated by weak dtRNAs at higher 3OC6HSL concentration (H_dR81 and H_dR82, FIGS. 3c and 10c, blue lines). To better explain our experimental data, we built a mathematical model for a positive feedback circuit regulated by dtRNAs and performed two-parameter bifurcation analysis on the system (for details see below). The result validates our data that dtRNAs with stronger stabilizing capability generate smaller bistable regions localized at low inducer concentrations, while weaker dtRNA regulation resulted in a larger bistable region shifted to high drug concentrations (FIG. 3d). Thus, this experiment illustrates the capacity of dtRNAs to fine tune gene circuit dynamics.

To explore the tunability dtRNAs offer for noncoding RNA levels, we built a CRISPR interference system to control small guide RNA (sgRNA) levels by redesigning the 5′ sequence of an sgRNA targeting a GFP promoter with dR1 and dR6, and two other top-performing dtRNAs (dR15 and dR19). When transcribed from a weak promoter, each redesigned sgRNA can guide dCas9 to bind with the cognate promoter region to inhibit downstream GFP expression (FIG. 3e). Stable sgRNAs are more likely to interact with dCas9 for stronger GFP inhibition. Indeed, we found that GFP fluorescence from cells expressing redesigned sgRNAs was significantly lower, yielding about 22% to 36% decrease compared to the original sgRNA lacking dtRNA stabilization (sgRNA_WT). These results demonstrate that noncoding RNA levels can also be tuned by synthetic dtRNAs (FIG. 3f).

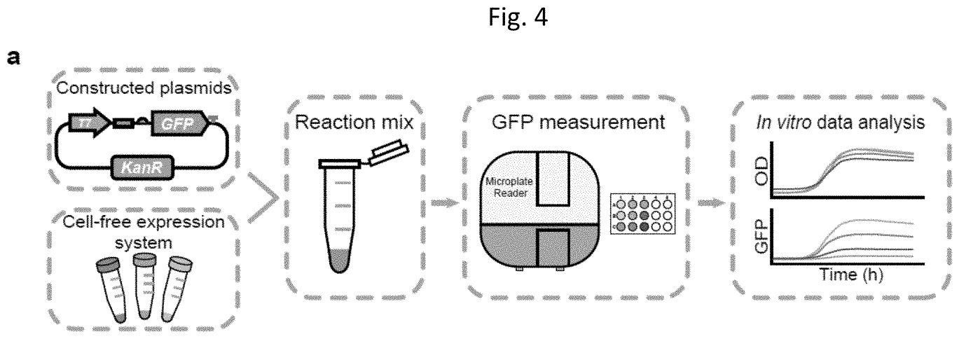

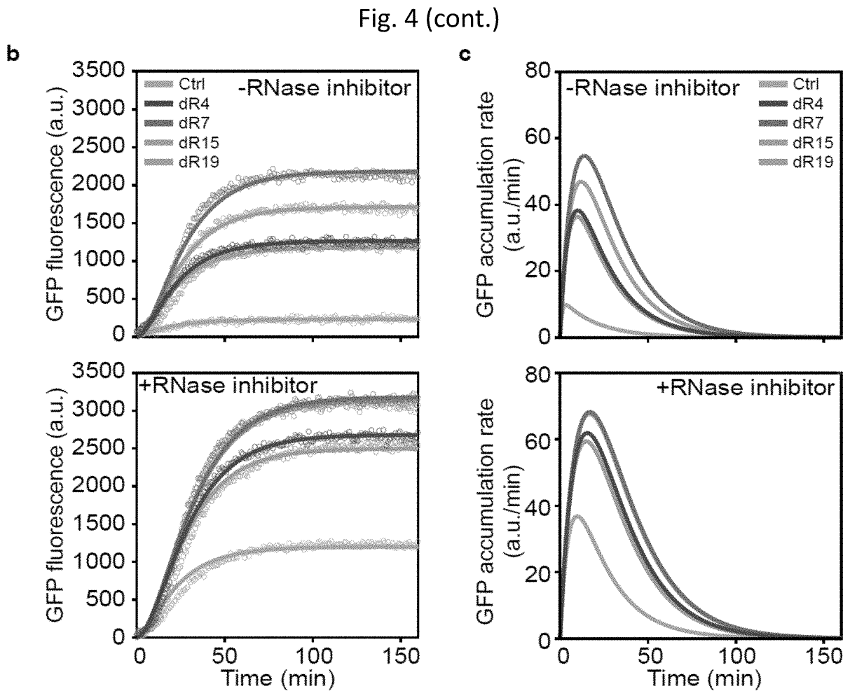

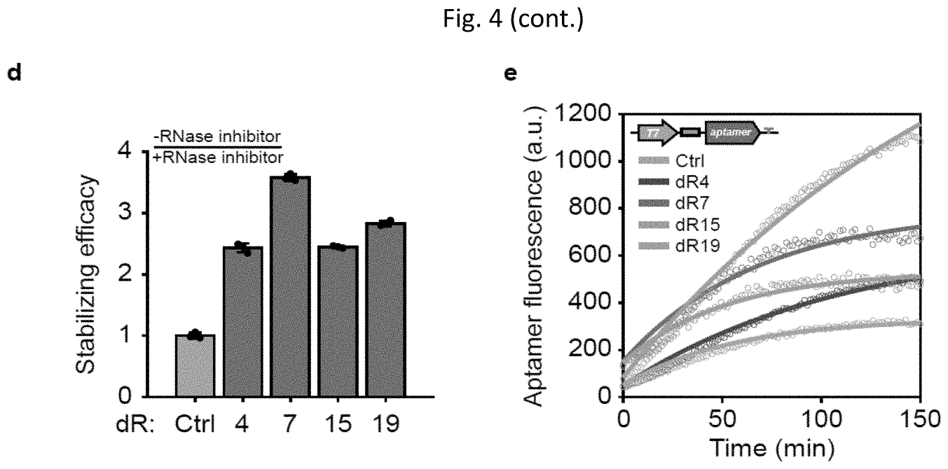

In vitro regulation of gene and RNA aptamer production by synthetic dtRNAs. Cell-free expression systems have been widely used in synthetic biology, metabolic engineering and in vitro diagnostics^{6,60-62}. To test whether synthetic dtRNAs enable regulation of gene expression in cell-free expression systems, we constructed two circuits with dtRNAs that showed good performance with sgRNAs (dR15 and dR19) along with two additional circuits with randomly selected top-performing dtRNAs (dR4 and dR7) to measure their impact on GFP expression in cell-free transcription-translation expression systems (FIG. 4a). For these experiments, triple guanines (GGG) were inserted at the 5′ end of the dtRNAs to ensure efficient transcription via T7 RNA polymerase.

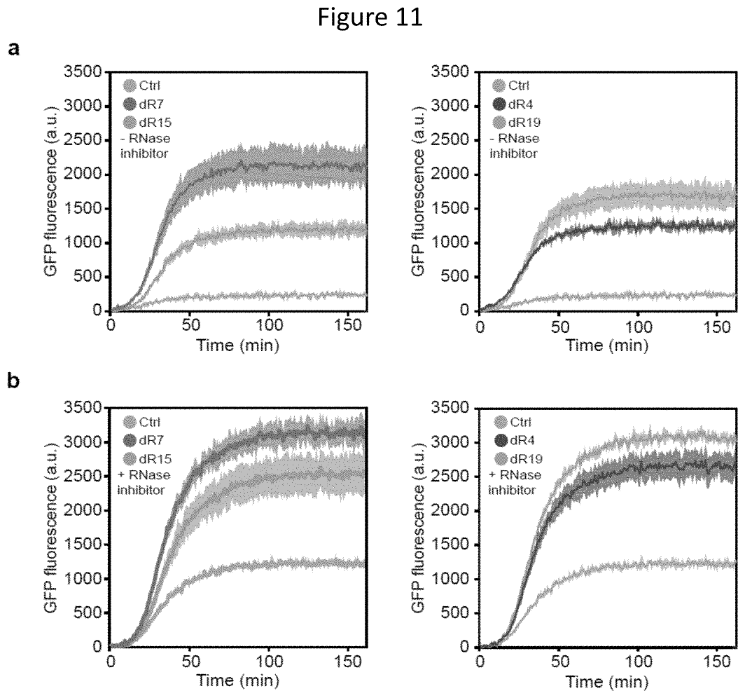

We first performed measurements without the addition of RNase inhibitor to each reaction (-RNase inhibitor group). The result in FIG. 4b (top) shows that GFP fluorescence of each circuit starts to increase shortly after the reaction begins, and it reaches a steady state after about an hour of reaction (FIG. 11a). Steady-state GFP fluorescence is much stronger for the circuits regulated by synthetic dtRNAs, where the circuit regulated by dtRNA dR7 displays about a 10-fold fluorescence enhancement. Enhancement effects can also be detected for each reaction with RNase inhibitor treatment (FIG. 4b, bottom and FIG. 11b). In both cases, the dtRNAs significantly increased GFP fluorescence compared to the control.

To better quantify gene expression enhancement due to RNA stability increases, we constructed a dynamic model to describe dtRNA-regulated GFP expression enhancement in both scenarios (FIG. 4b, solid lines). Since the cell-free system provides abundant molecular machinery for transcription and translation, we chose to use a simplified model that includes only these two steps without nonlinear terms. We solved this simplified model analytically and fitted against experimental time course directly. Fitting results gave us a more quantitative view of the efficacy of the dtRNAs and are consistent with experimental observations. Using model-fitted parameters, we can calculate GFP accumulation rates over time in both scenarios, where circuits regulated by dtRNAs display much faster GFP accumulation rates compared to the control (FIG. 4c). Theoretical derivations show that the time required for the GFP accumulation rate to reach its maximum (peak of the curve) is only dependent on the mRNA and protein degradation rates (Mathematical modeling). Given that protein degradation rates remain constant for all scenarios, the right-shifted peaks of dtRNAs mathematically support decreased mRNA degradation rates.

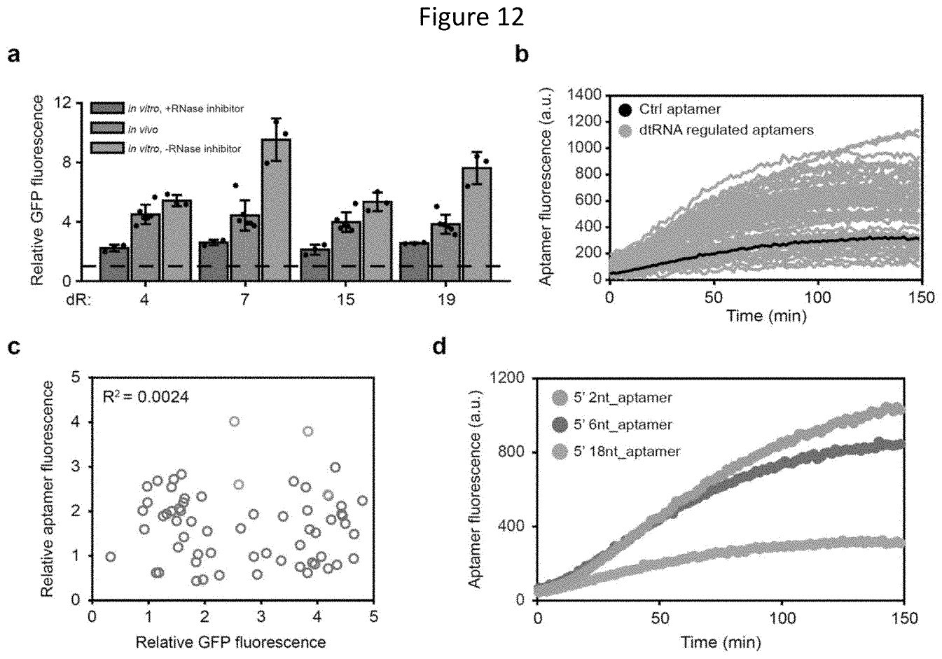

Stabilizing efficacy, defined as the ratio between the steady state GFP concentration without RNase inhibitor and with RNase inhibitor treatment, measures the robustness of dtRNAs in vitro against RNase activities, which could impact dtRNAs effectiveness (compare FIG. 4b top and bottom). It can be seen in FIG. 4d that all dtRNAs display over 2-fold stabilizing efficacy compared to the control. dtRNA dR7 yields the strongest enhancement at 3.6-fold, illustrating stability of dtRNAs even in the presence of RNase. The environmental dependence of the dtRNA's stability enhancement potential is further quantified by comparing relative GFP intensities in live bacteria cells or in cell-free expression systems (FIG. 12a). It can be seen that the dtRNA's stabilization capacity is most pronounced in vitro without RNase inhibitor.

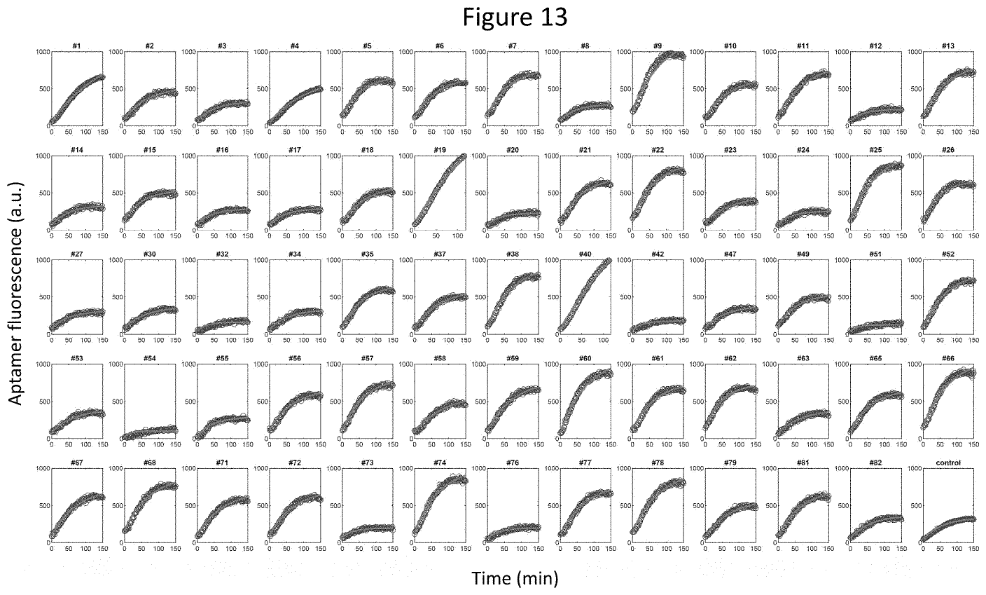

To further investigate the effect of dtRNA on RNA stability in vitro, we next coupled dtRNAs to the RNA aptamer Broccoli to directly measure whether dtRNAs can influence RNA levels in cell-free expression systems. 65 dtRNAs spanning the dynamic range of the library were selected, designed and ligated to the 5′ end of the Broccoli aptamers, and their fluorescence was measured using a plate reader. It can be seen that most of the dtRNAs significantly enhanced the aptamer fluorescence (FIG. 12b). However, we did not observe any significant correlations between in vivo GFP enhancement and cell-free aptamer regulation, probably due to different mechanisms between GFP expression in E. coli and in vitro aptamer transcription in their respective expression systems (FIG. 12c). One interesting finding is that dtRNAs of small size (3 bp or 6 bp stems) tend to strongly enhance aptamer fluorescence levels (FIG. 12c, green circles). In addition, to clearly show that dtRNAs can be used to directly manipulate Broccoli aptamer levels, we specifically compared four dtRNAs (dR4, dR7, dR15 and dR19) that were used to regulate in vitro GFP expression. We used a mathematical model to fit their experimental data (FIG. 13 and Supplementary information below). Interestingly, all four dtRNAs exhibited increased aptamer fluorescence in the cell-free expression system, out of which dR19 showed a nearly 4-fold enhancement compared to control (FIG. 4e). By fitting model parameters, we also calculated the half-life of each regulated aptamer (Table 5). These results demonstrate that dtRNAs can be applied to directly regulate RNA aptamer production with a wide dynamic range in cell-free systems.

TABLE 5
Information on estimated half-life of
dtRNA regulated Broccoli aptamers.

| dtRNA ranking | Half life (min) |
|---|---|
| 1 | 118.16 |
| 2 | 48.53 |
| 3 | 38.14 |
| 4 | 77.05 |
| 5 | 41.09 |
| 6 | 50.03 |
| 7 | 47.38 |
| 8 | 33.92 |
| 9 | 61.23 |
| 10 | 50.98 |
| 11 | 58.04 |
| 12 | 25.74 |
| 13 | 50.77 |
| 14 | 37.67 |
| 15 | 35.89 |
| 16 | 33.25 |
| 17 | 33.79 |
| 18 | 52.00 |
| 19 | 130.19 |
| 20 | 26.27 |
| 21 | 51.10 |
| 22 | 45.63 |
| 23 | 52.85 |
| 24 | 29.49 |
| 25 | 51.81 |
| 26 | 35.58 |
| 27 | 36.46 |
| 30 | 43.17 |
| 32 | 18.67 |
| 34 | 38.92 |
| 35 | 43.90 |
| 37 | 52.63 |
| 38 | 50.88 |
| 40 | 161.05 |
| 42 | 20.40 |
| 47 | 41.69 |
| 49 | 41.05 |
| 51 | 14.64 |
| 52 | 49.36 |
| 53 | 44.90 |
| 54 | 11.25 |
| 55 | 31.39 |
| 56 | 48.61 |
| 57 | 44.80 |
| 58 | 65.83 |
| 59 | 51.34 |
| 60 | 53.69 |
| 61 | 46.94 |
| 62 | 36.59 |
| 63 | 42.20 |
| 65 | 43.39 |
| 66 | 53.56 |
| 67 | 51.23 |
| 68 | 51.02 |
| 71 | 49.61 |
| 72 | 50.97 |
| 73 | 22.99 |
| 74 | 50.01 |
| 76 | 23.21 |
| 77 | 55.24 |
| 78 | 55.11 |
| 79 | 56.02 |
| 81 | 51.84 |
| 82 | 40.95 |
| Control | 38.56 |

Improved viral diagnostics using hybrid dtRNA/toehold switch sensors. The toehold switch is a programmable RNA device that can interact with a user-specified target RNA to activate translation of a protein of interest²⁰ and has been widely applied in areas including in vitro viral diagnostics^{6,63}, gene circuit engineering^{22,60,64} and education⁶⁵. Toehold switches feature a long single-stranded region known as a toehold at their 5′ end that is designed to initiate binding with the target RNA. However, transcripts with excessive 5′ single-stranded regions could be easily targeted and digested by RNases (FIG. 7c, d). This phenomenon was observed in our Broccoli aptamer assay where aptamers with a longer 5′ single-stranded region showed reduced fluorescence (FIG. 12d). To address this limitation, we coupled toehold switches with dtRNAs to improve their performance in a diagnostic assay. These hybrid systems were constructed by inserting dtRNAs at the 5′ end of an existing toehold switch designed for detection of norovirus in paper-based cell-free reactions (FIG. 5a). Five hybrid systems were designed using the main structure of the dtRNA with the best performance in in vitro gene expression measurements and the aptamer assay (dR19, FIGS. 4b and 4e). Hybrid systems were constructed with different combinations of 5′ spacing and insulator sequences: dR19_1 (2-nt 5′ spacing, 6-nt insulator), dR19_2 (2-nt 5′ spacing, 10-nt insulator), dR19_3 (2-nt 5′ spacing, 18-nt insulator), dR19_4 (6-nt 5′ spacing, 6-nt insulator) and dR19_5 (8-nt 5′ spacing, 6-nt insulator). The β-galactosidase (lacZ) α peptide (lacZα) was used as the reporter as previously described⁶³. This short peptide undergoes complementation with added β-galactosidase ω peptide during the in vitro translation reaction to generate an active enzyme and cleave a colorimetric reporter substrate.

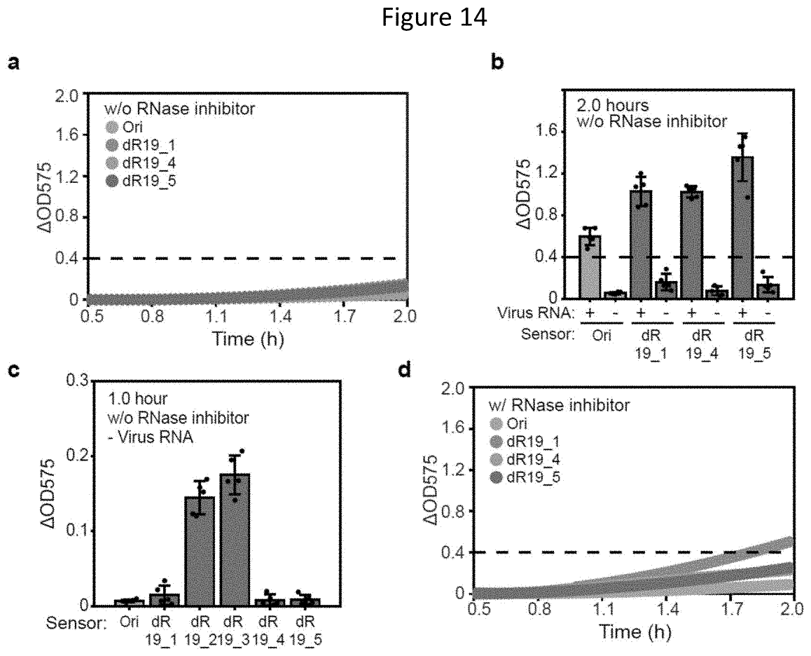

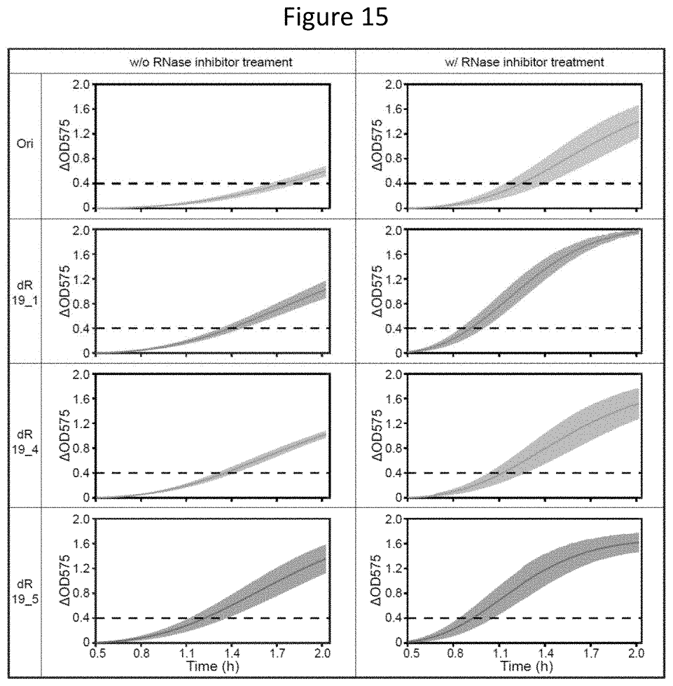

To test these hybrid sensors in paper-based diagnostic systems, synthetic norovirus RNA was introduced to paper-based devices containing cell-free reactions and DNA templates for transcription of the sensors without RNase inhibitor present. We observed that sensors with dtRNAs (dR19_1, dR19_4 and dR19_5) exhibited faster detection speed (1.22 hours, ΔOD575=0.4) without leaky expression, while the original sensor (Ori) without dtRNA only showed detectable signals after 1.74 hours of induction (FIG. 5b, top and FIGS. 14a, b). Sensor dR19_2 and dR19_3 exhibited leaky expression and thus were not subjected to further experiments (FIG. 14c). To test if the detection speed could be further improved, we proceeded to treat the paper-based device with RNase inhibitor for the second-round diagnostics. Remarkably, we found that all devices showed even faster detection speed against the group not treated with inhibitor, where signals of sensor dR19_1 and dR19_5 can be discerned within an hour (0.9 hour), indicating that the 5′ dtRNA structure can significantly improve the speed for viral diagnostics with RNase inhibitor treatment (FIG. 5b, bottom). At the same time, however, higher expression leakage is also observed for each device, indicating the addition of RNase inhibitor, although it accelerates reaction speed, can also increase the likelihood of false positive results (FIG. 14d). Further analysis demonstrates that non-inhibitor-treated sensor dR19_5 displays low expression leakage but faster diagnostic speed than the original sensor (Ori) in the presence of RNase inhibitor. Thus, hybrid sensors enhanced with dtRNAs can exceed the performance of standard toehold switch assays without requiring the addition of RNase inhibitor. From photographs and their corresponding diagnostic results, we confirm the improvement of viral diagnostics by using the hybrid dtRNA/toehold switch devices (FIGS. 5c, d). The details of each reaction can be found in FIG. 15.

### Discussion:

A great many methods have been developed to meet the increasing demand for precise and predictable control of gene expression. Naturally occurring RNA stabilizers or engineered 5′ stability hairpins that thwart RNase activity hold the potential to directly control RNA half-life and have been applied to regulate cellular RNA levels as well as heterologous protein yields⁴⁵⁻⁴⁸. In this study, we systematically identify the RNA structural features that influence stability, design a library of synthetic dtRNAs, and use them to tune gene expression levels in vivo and in vitro. We find that application of structure-stability relationships discerned from the library enables semi-quantitative predictions of the performance of newly designed dtRNAs. Moreover, we demonstrate multiple applications of dtRNAs by using them to increase the strength of CRISPR interference, tune gene circuit behavior and aptamer stability, and to enhance the speed and stability of paper-based viral diagnostics.

Previous studies have investigated 5′ stabilizing elements with an interest in increasing mRNA stability and understanding RNase substrate specificity^{46,48,50}, while others have designed 5′ UTRs to manipulate translation of mRNAs^{14,51}. Specifically, portable mRNA-stabilizing 5′-UTR sequences have been demonstrated to increase GFP mRNA stability⁵⁰. In this work, we engineered and tested a more comprehensive set of hairpins with systematically designed secondary structures that are able to not only tune RNA stability up, but also destabilize RNA molecules. Expanding earlier work of analyzing free energy (ΔG) of hairpin designs⁴⁸, we systematically explored the structural feature space with the aim to elucidate dtRNA's structure-stability relationships. Our results demonstrate that 5′ UTR RNA secondary structure can be engineered with varying features such as stem-loop length, sequence context and RNase cleavage sites to achieve wide dynamic range over RNA stability regulation, in turn allowing precise control over gene expression and non-coding RNA activity. Moreover, compared to engineered synthetic promoter and RBS libraries, it is relatively easy to construct dtRNAs following our design rules in diverse engineering scenarios. Similar to previous studies, our work also confirms that gene expression regulation by dtRNA modules exert little effect on cell growth, indicating that compared to the other gene expression regulation methods, RNA manipulation renders less burden for cell economy (FIG. 2e, inset)^{50,66,67}. Both locations and features of the dtRNAs structures interact with aspects of translation and degradation processes to affect stability of different types of RNAs, including mRNAs, guide RNAs, and toehold RNAs. Furthermore, our modest success in predicting dtRNA stabilizing capabilities suggests the possibility of fully designing dtRNAs in silico when enough nearby RNA secondary context is taken into consideration (FIG. 9b). The utility of such dtRNA libraries combining high dynamic range with fine gradations in output is demonstrated in applications such as changing output behavior of synthetic circuits and viral diagnostics.

When assessing mRNA lifetime, it is important to note that degradation and translation are closely intertwined processes. Thus, only considering one to determine the final protein yield could overestimate the capabilities of dtRNAs. After being transcribed, mRNA is competitively targeted by RNases and ribosome subunits, where, in theory, a stable mRNA has a higher chance for ribosome binding than unstable mRNA. Furthermore, highly translated genes can also be shielded by active ribosomes that serve to protect against RNase activities. This positive side effect of enhanced RNA stability can be observed in our RT-qPCR results where RNA fold increase can account for over 94% but still not all GFP expression increases (FIG. 9a). Therefore, stabilized RNAs could possess mildly higher translation rates than the unstable ones.

We also applied our dtRNA modules to directly upregulate gene expression and tune RNA aptamer levels in cell-free expression systems with a 10-fold dynamic range. An RNA-based device, the toehold switch sensor, is optimized with our dtRNAs for rapid paper-based viral diagnostics. Higher detection sensitivity with low expression leakage is achieved using the redesigned sensors, making them more compatible for potential field-ready diagnostics. More importantly, dtRNA robustness against RNase activities suggests that they can also be used to enhance expression in crude-extract-based cell lysates, which are substantially cheaper to produce but have higher RNase levels^{68,69}. Previous work has shown that native 5′ UTR structures can be used to enhance gene expression in such cell-free reactions⁷⁰. Overall, our work provides a purely RNA-based method to regulate gene expression in vivo and in vitro that can be used for a variety of different biotechnological applications.

### Experimental Methods:

Strain, media and culture condition. All molecular cloning experiments were performed in Escherichia coli DH10B (Invitrogen). Synthetic circuits (FIG. 3) were tested in E. coli K-12 MG1655 with Cells were grown at 37° C. in liquid and solid Luria-Bertani (LB) broth medium with 100 μg/mL ampicillin or 50 μg/mL kanamycin, and were shaken in 5-mL or 15-mL tubes at 220 rotations per minute (rpm). The chemical 3OC6HSL was dissolved in ddH₂O and were further diluted to various working concentrations for dose-response and hysteresis measurements.

Plasmid construction. Most genes were obtained from iGEM Registry (http://parts.igem.org/Main_Page). Plasmids were constructed based on general molecular biology techniques and standardized Biobrick cloning methods as previously describer. For example, to assemble GFP gene (E0040) with a strong RBS (B0034), plasmids with GFP gene were digested with xbaI and PstI as the cloning insert while plasmids containing RBS were digested with SpeI and PstI as the cloning vector. Digested plasmids were then separated on 1% TAE Agarose gel by gel electrophoresis. Gel bands with correct insert or vector size were selected and purified using the PureLink gel extraction Kit (Invitrogen). Gel extraction products with insert and vector were ligated by T4 DNA ligase (New England Biolabs, NEB) and transformed into E. coli DH10B. Transformed cells were plated on LB agar plates with 100 μg/mL ampicillin, or 50 μg/mL kanamycin for screening. In the end, plasmids extracted by GenElute HP MiniPrep Kit (SIGMA-ALDRICH) were confirmed through gel electrophoresis (digested by EcoRI and PstI) and Sanger DNA Sequencing (Biodesign Sequencing Core, ASU). Similar Biobrick cloning steps were taken for the following genetic components until the entire circuit has been constructed. All names and Biobrick number of genetic components can be found in Table 1.

TABLE 1
Information on iGEM Registry of standard biological components
and commonly used genetic parts related to the figures.

| Biobrick number | Abbreviation | |
|---|---|---|
| or gene name | in the paper | Gene description |
| BBa_J23104 | CP | Constitutive promoter family member |
| | | used in FIG. S1 and FIG. 3 |
| BBa_J23116 | CP | Constitutive promoter family member |
| | | used in FIG. 1, FIG. 2, and FIG. 3 |
| BBa_J23105 | CP | Constitutive promoter family member |
| | | used in FIG. S3e |
| BBa_J23109 | CP | Constitutive promoter family member |
| | | used in FIG. S3e |
| BBa_R0062 | pLux | LuxR activated promoter in concern |
| | | with HSL used in FIG. 3 |
| T7 promoter | T7 | T7 polymerase specific promoter used |
| | | in FIG. 4, FIG. 5 and FIG. S7-S10 |
| BBa_B0034 | RBS | Ribosome binding site used in FIG. 1-4 |
| BBa_B0031 | RBS | Ribosome binding site used in FIG. S3f |
| BBa_B0032 | RBS | Ribosome binding site used in FIG. S3f |
| BBa_B0015 | T | Transcriptional terminator used for |
| | | engineering all the circuits |
| BBa_E0040 | GFP | Green fluorescence protein used as the |
| | | reporter |
| BBa_E1010 | mRFP | Red fluorescence protein used as the |
| | | reporter in FIG. S3c and S3d |
| BBa_C0062 | LuxR | LuxR activator used in FIG. 3 |
| BBa_pSB1A3 | Vector | Plasmid backbone used for circuit |
| | | cloning in FIG. 1 and FIG. 2 |
| BBa_pSB3k3 | Vector | Plasmid backbone used for circuit |
| | | cloning in FIG. 3 |
| pCOLODuet | Vector | Plasmid backbone used for in vitro |
| | | experiments in FIG. 4 and FIG. 5 |

For construction of the circuits with dtRNAs or sgRNAs, each structure was analyzed and designed by the NUPACK design package⁷² and their respective DNA oligos were synthesized by IDT. Biobrick XbaI and PstI cleavage sites were added at 5′ or 3′ end of the DNA oligos. DNA Oligos for the same dtRNA were diluted with ddH₂O and hetero duplexed on a heat block and were further ligated into the plasmids with the promoter digested by XbaI and PstI. The guide sequence of sgRNA or redesigned sgRNAs were designed and then synthesized by IDT. The sequence 5′-GCTA-3′ and 5′-AAC-3′ were added on sgRNA forward and reverse primers, respectively. DNA oligos for the same sgRNA were diluted by ddH₂O, hetero duplexed on a heat block and ligated to the vector digested by SapI as previously describer. The rest of the cloning steps remain the same as the general gene circuit construction.

Plate reader OD and fluorescence measurements. All sequencing-confirmed gene circuits were transformed into E. coli DH10B. Single colonies were picked and cultured in 4 mL of LB medium with 100 μg/mL ampicillin. Cells were shaken until they were evenly distributed in the medium of which 300 μL were transferred into 96-well plate for OD and fluorescence measurements. Optical density (OD600) and fluorescence (excitation: 485 nm; emission: 530 nm) were measured every 15 minutes at 37° C. under continuous plate shaking (Synergy H1 Hybrid Reader, BioTek) at 220 rpm over 21 hr. For all the experiments, at least three random colonies were picked as biological replicates. For stable protein expression, we chose the 16-hour data point for further analysis in the study unless specified.

Flow cytometry measurements. We used an Accuri C6 flow cytometer to perform the flow cytometry measurements (Becton Dickinson). Cultured samples were collected and run through the flow cytometer. For each sample, 20,000 individual cells were analyzed at the slow flow rate and the fluorescence intensity was not normalized with the cell density because it only measured single cell data. All the results were then collected in log mode and further analyzed by MATLAB (MathWorks).

RT-qPCR. For selected gene circuits, three biological replicates were used to quantify the mRNA levels. Total RNA was extracted from the 2 mL of cell culture using the Quick-RNA Fungal/Bacterial Miniprep Kit (Zymo Research). Purified RNA was treated in column with DNaseI (Zymo Research) to remove the extra DNA. Total RNA was eluted by nuclease-free water and the concentration quantified for the following experiments. cDNA was then synthesized from each RNA sample using iScript Reverse Transcription Supermix for RT-qPCR (Bio-Rad). For each 20-uL reaction, about 1 μg RNA was used for reverse transcription. qPCR was performed for each cDNA sample using iTaq Universal SYBR Green Supermix (Bio-Rad) and the experiment reaction was detected using the iQ5 Real-Time PCR detection system (Bio-Rad). Specifically, each cDNA sample contains an extra technical replicate, the total reaction volume for each sample is 10 μL and prokaryotic 16S rRNA was set as the endogenous control. We used previous reported primers (IDT) for both 16S rRNA and GFP amplification³². The sequence of primers for 16S rRNA are 5′-GAATGCCACGGTGAATACGTT-3′ (SEQ ID NO:83) (rrnB, forward, starting at the 1361st nucleotide), and 5′-CACAAAGTGGTAAGCGCCCT-3′ 3′ (SEQ ID NO:84) (rrnB, reverse, starting at the 1475th nucleotide) and the sequence of GFP primers are 5′-CAGTGGAGAGGGTGAAGGTGA-3′ (SEQ ID NO:85) (forward, starting at the 87th nucleotide); and 5′-CCTGTACATAACCTTCGGGCAT-3′ (SEQ ID NO:86) (reverse, starting at the 283th nucleotide). Bio-rad CFX Manager software version 3.1 was used to analyze the data. To investigate the fold change over mRNA levels, we averaged each Cₜ value of 16S rRNA and GFP with their biological replicates and calculated the delta Cₜ based on Cₜ^{target}−Cₜ^{16S}. Fold change for each sample was further calculated according to the biological control (circuit without dtRNA regulation) by 2^{-(ΔΔCt)}. The minimum information for publication of quantitative real-time PCR (MIQE) is also provided in Table 2.

TABLE 2
Minimum Information for Publication of Quantitative Real-Time PCR (MIQE).
Experimental Design

| | |
|---|---|
| Definition of experimental and control | Experimental group: GFP mRNA levels for |
| groups | circuit with naturally occurring or synthetic |
| | dtRNA library regulation in FIG. 1 and FIG. S3; |
| | Control group: GFP mRNA levels for circuits |
| | without stabilizer regulation. |
| Number within each group | Experimental group: 9; Control group: 1. |
| Assay carried out by the core or | Reverse transcription and qPCR experiments |
| investigator's laboratory? | were performed in the synthetic biology lab of |
| | SBHSE at Arizona State University. |
| Acknowledgement of authors' | See Author Contributions statement |
| contributions | |

Sample

| | |
|---|---|
| Description | Growing E. coli cell cultures (fresh) |
| Volume/mass of sample processed | 2.0 mL |
| Microdissection or macrodissection | Not applicable |
| Processing procedure | |
| If frozen - how and how quickly? | No frozen |
| If fixed - with what, how quickly? | No fixed |
| Sample storage conditions and duration | Fresh sample, no storage |
| (especially for FFPE samples) | |
| Procedure and/or instrumentation | Quick-RNA Fungal/Bacterial Miniprep |
| | Instruction Manual |
| Name of kit and details of any | Quick-RNA Fungal/Bacterial Miniprep Kit |
| modifications | (ZYMO RESEARCH), Cat. #: R2014 |
| Source of additional reagents used | 100% Ethanol, 200 PROOF PURE ETHONAL |
| | (V1001) |
| Details of DNase or RNase treatment | DNase 1 w/ DNA digestion buffer 250 U, Cat. # |
| | E1010 |
| Contamination assessment (DNA or RNA) | PCR amplification of GFP fragment for total- |
| | RNA before and after DNase treatment. |
| Nucleic acid quantification | Spectrophotometry |
| Instrument and method | BioTek's Synergy H1 multi-mode Reader |
| Purity (A260/A280) | 1.93~2.03 |
| RNA integrity method/instrument | Running agarose gel to check RNA quality |
| RIN/RQI or Cq of 3′ and 5′ transcripts | Not applicable |
| Inhibition testing (Cq dilutions, | Not applicable |
| spike or other) | |

Reverse Transcription

| | |
|---|---|
| Complete reaction conditions | 25° C. 5 min for priming, then 46° C. 20 min |
| | for reverse transcription, and 95° C. 1 min |
| | for inactivation, 4° C. forever. |
| Amount of RNA and reaction volume | Total ~1 μg RNA, 20 μL volume for reaction |
| Priming oligonucleotide (if using GSP) | Random primers |
| and concentration | |
| Reverse transcriptase and concentration | Reverse Transcription Supermix |
| Manufacturer of reagents and catalogue | iScript cDNA Synthesis Kit, Bio-Rad, |
| numbers | Cat. #1708841 |
| Storage conditions of cDNA | −80° C. refrigerator |

qPCR Target Information

| | |
|---|---|
| Target gene | GFP (16S rRNA was used as the endogenous |
| | gene) |
| Sequence accession number | Not applicable |
| Location of amplicon | +87~+283 of GFP gene |
| Amplicon length | 197 bp |
| In silico specificity screen (BLAST, | Designed using Primer-BLAST, NCBI |
| and so on) | |
| Pseudogenes, retropseudogenes, or other | None |
| homologs? | |
| Location of each primer by exon or | Not applicable |
| intron | |
| What splice variants are targeted? | Not applicable |

qPCR Oligonucleotides

| | |
|---|---|
| Primer sequences | 16s rRNA Forward: |
| | 5′-GAATGCCACGGTGAATACGTT-3′ |
| | (SEQ ID NO: 83) |
| | 16s rRNA Reverse: |
| | 5′-CACAAAGTGGTAAGCGCCCT-3′ |
| | (SEQ ID NO: 84) |
| | GFP Forward: |
| | 5′-CAGTGGAGAGGGTGAAGGTGA-3′ |
| | (SEQ ID NO: 85) |
| | GFP Reverse: |
| | 5′-CCTGTACATAACCTTCGGGCAT-3′ |
| | (SEQ ID NO: 86) |
| Location and identify of any | None |
| modifications | |
| Manufacturer of oligonucleotides | Integrated DNA Technologies (IDT) |

qPCR Protocol

| | |
|---|---|
| Complete Reaction Condition | Samples were first denatured at 95° C. for |
| | 2 mins, followed by 40 cycles, 95° C. |
| | denaturation and 60° C., 30 s annealing, |
| | extension and fluorescence reading. |
| Reaction volume and amount of | 10 μL total reaction volume; 0.1 μL cDNA |
| cDNA/DNA | |
| Primer, (probe), Mg++ and dNTP | All detailed information can be found in iTaq |
| concentrations | Universal SYBR Green Supermix, Bio-rad, Cat. |
| | #172-5120 |
| Polymerase identity and concentration | |
| Buffer/kit identity and manufacturer | |
| Additives (SYBR Green 1, DMSO, etc.) | SYBR Green 1 |
| Manufacturer of plates/tubes and | Bio-rad, Cat. #HSP3801 |
| catalog number | |
| Complete thermocycling parameters | 95° C. for 2 mins; 40 cycles of 95° C. 5 s; |
| | 60° C. for 30 s |
| Reaction setup (manual/robotic) | Manual |
| Manufacturer of qPCR instrument | iQ5 Real-Time PCR detection system |
| | (CFX384TM, Bio-Rad) |

qPCR Validation

| | |
|---|---|
| Specificity (gel, sequence, melt, or | Primer pairs were designed for GFP and 16S |
| digest) | rRNA |
| For SYBR Green 1, Cq of the NTC | 40 |
| PCR efficiency calculated from slope | 93.82% |
| Calibration curves with slope and | Cq values: 23.1907079; 26.1641088; |
| intercept | 29.6333439; 33.1484379; 37.0967272 |
| | Slope: −3.4796; Intercept: 22.887 |
| | (1:10 dilution) |
| R² of standard curve | 0.99757 |
| Linear dynamic range | Dilutions spanned four orders of magnitude |

Data Analysis

| | |
|---|---|
| qPCR analysis program (source, version) | Bio-rad CFX Manager software Version 3.1 |
| Method of Cq determination | Auto threshold |
| Outlier identification and disposition | None |
| Justification of number and choice of | 16S rRNA was used as the reference which |
| references | consistently express in log phase of E. Coli |
| genes | cells |
| Description of normalization method | Each sample was first normalized to 16S rRNA |
| | reference to calculate the delta Cq. Samples |
| | then normalized to the experimental control |
| | to calculate the fold change |
| Number and concordance of biological | Three biological replicates for each sample |
| replicates | |
| Number and stage (RT or qPCR) of | Two technical replicates |
| technical replicates | |
| Repeatability (intra-assay variation) | Standard deviation between three biological |
| | replicates of each sample can be found in FIG. |
| | 1C and FIG. S4a |
| Statistical methods for result | None |
| significance | |

Note:
Same primers for GFP and 16S rRNA target were used as previously described3.

Hysteresis experiments. We used our previously reported protocol to perform the hysteresis experiments³³. In detail, gene circuits of the synthetic positive feedback loop were constructed in a low-copy plasmid and transformed into E. coli K-12 MG1655 strain with lacI−/−. Single colonies for three replicates were picked for each sample and cultured at 37° C., 220 rpm overnight in LB medium with 50 μg/mL kanamycin. For OFF-ON experiments, overnight cultured cells (initial OFF cells) were diluted into fresh LB medium at a 1:100 ratio and distributed into 5-mL polypropylene round-bottom tubes (Falcon) with various 3OC6HSL concentrations. Fluorescence of each sample was measured using an Accuri C6 flow cytometer (Becton Dickinson). In our experiments, GFP fluorescence became stable after ˜12 hours of induction. For ON-OFF experiments, cells were first induced by 2 nM 3OC6HSL for 12 hours to ensure the fully induction as the initial ON state. These ON state cells were then collected through low speed centrifugation, washed once and further diluted to the fresh LB medium at 1:100 ratio. Various 3OC6HSL concentrations were then added to each sample for culture. Flow cytometry measurements were performed at 12 and 16 hours, respectively. We used 16-hour results as the ON-OFF dataset in FIG. 3 and FIG. 10.

RNA aptamer assay. Sequences of dtRNA-regulated Broccoli aptamers were designed using NUPACK and were further synthesized from IDT. T7 promoter and terminator sequences were inserted to each redesigned aptamer through PCR. Amplified double-stranded DNA molecules were purified using MinElute PCR purification kit (QIAGEN) and measured their concentration via Nanodrop spectrophotometer. Purified DNA was then diluted and mixed with cell-free transcription-translation systems (PURExpress, NEB). Each sample with 4 uL reaction mix was loaded to the 384 well plate for a five-hour plate reader measurement, and the fluorescence of each sample reached the peak value after about two-hour incubation at 37° C. In this experiment, we used a 30-nM DNA concentration for each sample for the reactions and the fluorescence was measured every 90 seconds.

Hybrid dtRNA/toehold sensor plasmid construction. Synthetic DNAs encoding the redesigned norovirus-specific toehold sensors were synthesized by IDT. All cloning steps are following the general molecular biology technologies. Synthetic DNAs were amplified by PCR and inserted into the plasmid backbone using Gibson assembly⁷⁴. Complete plasmids were further confirmed by Sanger sequencing (Biodesign Sequencing Core, ASU). Plasmids and primers were described previously⁶³.

Paper-based cell-free systems preparation. The protocols used for the paper-based cell-free reactions have been described previously⁶³. Briefly, cell-free transcription-translation systems (PURExpress, NEB) were used to prepare the freeze-dried samples. The volume for each component of the reaction sample is 40% of cell-free solution A, 30% of cell-free solution B, 2% RNase inhibitor (Roche, 03335402001, distributed by MilliporeSigma) if needed, 2.5% chlorophenol red-b-D-galactopyranoside (Roche, 10884308001, distributed by MilliporeSigma, 24 mg/mL) and the remaining volume for toehold sensor DNA, lacZω and nuclease-free water. The final concentration for the synthetic DNA plasmid of each paper device is 30 ng/μL. The paper for the assays was first cut to a 2-mm diameter using a biopsy punch and transferred into PCR tubes. The prepared cell-free reaction mix (1.8 μL for each device) was then added into the PCR tubes with the paper disks and flash frozen in liquid nitrogen. Frozen devices were transferred to a lyophilizer to freeze-dry overnight. Completely dry paper devices were ready for use as viral diagnostics and can be stored at room temperature as previously described^{60,63}.

### Design Methods:

This section describes the method for in silico design of the synthetic dtRNA library through NUPACK design package¹. The same method is also used to design new dtRNAs for in vitro gene expression regulation and toehold sensor optimization for paper-based viral diagnostics.

Definition of dtRNA secondary structure domains. We first specify the secondary structure domains of the dtRNA library. A single hairpin is set to be the basic structural frame for each dtRNA. As shown in FIG. 2, factors such as the 5′ spacing, stem length and the number of GC pairs, and loop size are considered for structure optimization. Based on these features, we define the 5′ spacing region as domain “a”; the stem and loop of the hairpin frame as domains “b” and “c”, respectively; the 10-nt insulator sequence as domain “d”; and the rest of the downstream sequences are defined as domain “e”. Previous research has demonstrated that gene expression is significantly correlated with the folding energy from the RBS region to +38 nt of the coding sequence^{2,3}. Accordingly, we select 64 nt as the downstream sequence, which contains the RBS region (e.g., strong RBS BBa_B0034 in FIG. 2: AAAGAGGAGAA (SEQ ID NO:87)) and the first 38 nt of the GFP gene (Table 51, BBa_E0040). For T7-promoter-induced gene expression (FIGS. 4 and 5), a GGG leader sequence is inserted at the beginning of 5′ spacing (domain “a”) for efficient transcription.

Optimization of NUPACK scripts and dtRNA library sequence generation. After completing definition of the domains of the dtRNA structure, NUPACK scripts are needed to generate the sequence to fit the design principles. We first determined the basic settings for the design: the material is chosen to be RNA; the temperature is set at 37° C. and the trial number is set as 10 which indicates the number of independent sequences to perform for one-time NUPACK design (Maximum 10).

We then define the base structure of each dtRNA in the library. In particular, we use DU+ notation to specify the single-stranded or base-paired nucleotides: U denotes the single-stranded nucleotides and D denotes the base-paired nucleotides. To define a hairpin structure with a 4-bp stem and 4-nt loop, for example, the algorithm format should be “D4 U4”. Accordingly, the general format for the dtRNA structure with a 6-nt 5′ spacing, 12-bp stem, 6-nt loop, 10-nt insulator sequence, and 64-nt downstream sequence is “U6 D12 U6 U10 U64”. Specifically, for designs with an imperfect hairpin structure such as the introduction of a bulge within the stem region, we use brackets to specify the structural hierarchies. For example, “D3 (U3 D3 U6 U3)” denotes the structure with 9 bp stem interrupted by 3-nt symmetrical bulge. To ensure each domain will not interfere with the others, we maintain all sequences to be single stranded except the dtRNA hairpin structure during design process.

We next assign specific sequences to each domain. If the assigned sequence is not specified or needs the NUPACK design package to determine, we use the letter “N” to denote these nucleotides. Otherwise, using A, U, C and G to represent the four ribonucleotides. For example, a script with dtRNA=U6 D12 U6 U10, dtRNA.seq=a b c b* d (b* represents the complementary sequence to b), domain a=UCUUCC, domain b=N3UCUUCCN3, domain c=UCUUCC and domain d=N10 represent a dtRNA with three RNase E cleavage sites UCUUCC inserted into 6 nt 5′ spacing (domain “a”), the middle 6 bp of the stem (domain “b” and “b*”), and in the 6-nt loop (domain “c”) while keeping the other nucleotides random.

For the final output of the synthetic dtRNA library, we choose Serra and Turner, 1995 as the basic RNA energy parameters and use 1.0 M Na⁺ and 0 M Mg²⁺ for the design algorithm⁴. To prevent runs of nucleotides or pairs of nucleotides, the following sequences were disallowed in the resulting designs: AAAAA, CCCCC, GGGGG, UUUUU, KKKKKK, MMMMMM, RRRRRR, SSSSSS, WWWWWW, YYYYYY.

Analysis and removal of unwanted designs. NUPACK design package calculates each design with a specific normalized ensemble defect which indicates the average percentage of incorrectly paired nucleotides at equilibrium relative to the design secondary structure which is evaluated by the Boltzmann-weighted ensemble of (unpseudoknotted) secondary structure. The best normalized ensemble defect is 0%, while 100% is the worst. We select the designs with the lowest normalized ensemble defect while removing the others to select the seed dtRNAs for each design criteria listed in FIG. 2. These seed dtRNAs are further analyzed by NUPACK to make sure no interaction occurs between the dtRNA structure and the selected downstream sequences as shown in NUPACK structure predictions. Additionally, selected dtRNAs should keep a downstream sequence identical to their original structures. Seed dtRNAs that do not meet the specified criteria are removed from the designs. To prevent the introduction of transcriptional terminator sequences, insulator sequences with rU residues are fully removed from consideration. Based on this analysis, we chose AAAACCAAAA (SEQ ID NO:88) as the general insulator sequence for each dtRNA design unless otherwise specified. Fluorescence measurement result show that the insulator sequence has a minor impact on GFP fluorescence (dR69).

The same method is used to denote the feature of dtRNAs to regulate gene expression in vitro and hybrid toehold sensors for viral diagnostics. In short, we select the desirable hairpin from the dtRNA library as the basal structure and define new 5′ spacing and insulator sequence as the design required (e.g., add GGG at the beginning of 5′ spacing for T7 promoter transcription preference). All designed dtRNAs are further analyzed and finalized as described above.

Examples of the Scripts for dtRNA Design:

#

# Basic Settings of dtRNA structure design

material=rna

temperature=37.0

trials=10

sodium=1.0

#

#

# Basic Sequence information

# Rnase E cleavage site=UCUUCC

# Common 3′ end sequence (RBS to first 38 nt of GFP sequence, total 64 nt)=TACTAGAGAAAGAGGAGAAATACTAGATGCGTAAAGGAGAAGAACTTTTCACTGGAGTTGTCCC (SEQ ID NO:89)

# Common 3′ end sequence structure=U13 D3 (U2 D3 (U1 D4 (U1 D2 (U3 D4 U8 U1)) U1) U1)

#

#

# dtRNA Structure Design

# example of dtRNA DU+ notation design of 6 nt 5′ spacing, 12 bp stem 6 nt loop with 10 nt insulator

structure dtRNA=U6 D12 U6 U10 U64

#

#

# Sequence denotation of each dtRNA domain

| | |
|---|---|
| domain a = N6 | # 5′spacing domain |
| domain b = N12 | # dtRNA Stem region |
| domain c = N6 | # dtRNA Loop region |
| domain d = N10 | # Insulator sequence |
| domain e = | |

TACTAGAGAAAGAGGAGAAATACTAGATGCGTAAAGGAGAAGAACTTTTCACTGGAGTTGTCCC (SEQ ID NO:89) #64 nt Common 3′ end sequence

#

#

# Define each domain of dtRNA structure

dtRNA.seq=a b c b* d e

#

#

# Following sequence patterns are disallowed to prevent runs of nucleotides or pairs of nucleotides

prevent=AAAAA, CCCCC, GGGGG, UUUUU, KKKKKK, MMMMMM, RRRRRR, SSSSSS, WWWWWW, YYYYYY

#

#

# Output=qualified dtRNA sequence

#

### Mathematical Methods:

Mathematic modeling for positive feedback circuit analysis. We constructed a mathematical model to clarify the underlying mechanism of the dynamic changes of a positive feedback circuit regulated by dtRNAs. We used a 2D ordinary differential equation (ODE) describing the transcription and translation process:

$$\frac{dM}{dt} = {v}_{0} + {v}_{1} \frac{{R}_{f}^{2}}{{R}_{f}^{2} + {K}_{B}} - \frac{{δ}_{M}}{α} M \qquad [ Eq1 ]$$

$$\frac{{dR}_{T}}{dt} = \frac{M}{M + {K}_{M}} {v}_{2} - {δ}_{R} {R}_{T} \qquad [ Eq2 ]$$

$$where$$

$${R}_{f} = {R}_{T} \frac{{L}^{n}}{{L}^{n} + {K}_{L}^{n}} \qquad [ Eq3 ]$$

[Eq1] describes the luxR mRNA transcription and degradation process. M is the abundance of luxR mRNA. v₀ stands for leakage transcription rate of lux promoter without binding of LuxR, while

$${v}_{1} \frac{{R}_{f}^{2}}{{R}_{f}^{2} + {K}_{B}}$$

represents the transcription rate with [LuxR-3OC6HSL]2 complex bound to the lux promoter, given in Hill Equation form⁵. v₁ is the maximum transcription rate when all lux promoters are fully bound by [LuxR-3OC6HSL]2. R_{f} stands for functional LuxR protein abundance that are activated through binding with 3OC6HSL. K_{B} is the square of the dissociation constant of lux promoter and [LuxR-3OC6HSL]₂ binding. The mRNA degradation process is given by a linear form

$$\frac{{δ}_{M}}{α} M .$$

δ_{M} is the degradation rate without dtRNA. The effect of dtRNA is measured by α, the relative dtRNA strength. α=1 if there is no dtRNA regulation. α>1 if dtRNA stabilizes mRNA and thus increases protein expression, while α<1 if dtRNA facilitates mRNA degradation and thus decreases protein expression.

[Eq2] describes LuxR protein translation and degradation process. R_{T} is total LuxR protein concentration in system, including free LuxR and LuxR bound with 3OC6HSL and/or lux promoter. The mRNA translation is given by a Michaelis-Menten kinetics form

$$\frac{M}{M + {K}_{M}} {v}_{2} ,$$

where v₂ is the maximum translation rate and K_{M} is the Michaelis-Menten constant, i.e. mRNA abundance when translation rate reaches half of maximum value v₂. LuxR protein degradation takes simple linear form δ_{R}R_{T}, where δ_{R} is the degradation rate of LuxR protein.

The relationship of total LuxR abundance R_{T} and functional LuxR abundance R_{f} is given by [Eq3] in Hill Equation form⁶. L stands for concentration of 3OC6HSL. n describes the cooperativity of 3OC6HSL-LuxR binding. K_{L} is the dissociation constant of 3OC6HSL-LuxR binding.

Using these ODE equations, we analyzed the dynamics of the self-activation system with XPPAUT (XPPAUT 8.0 January 2016)⁷. Parameter values used during analysis are shown in Table 4. A two-parameter bifurcation regarding α and L is performed (FIG. 3d). As we can see, the bistable region shifts to the low drug concentration as the increase of dtRNA strength (α).

TABLE 4
Information on parameters for mathematical modeling
of positive feedback circuit regulated by dtRNAs.

| Parameters | Description | Value | Source |
|---|---|---|---|
| ν₀ | Leakage transcription rate | 0.08 | Ref. 6 |
| | without [LuxR-3OC6HSL]₂ | | |
| | binding (min⁻¹) | | |
| ν₁ | Transcription rate when | 1.8 | Ref. 6 |
| | fully bound by [LuxR- | | |
| | 3OC6HSL]₂ (min⁻¹) | | |
| ν₂ | Maximum translation rate | 16 | Estimated |
| | (min⁻¹) | | |
| δ_{M} | mRNA degradation rate | 1.5 | Estimated |
| | without dtRNA (min⁻¹) | | |
| δ_{R} | LuxR protein degradation | 0.01 | Ref. 6 |
| | rate (min⁻¹) | | |
| K_{B} | Square of dissociation | 0.8 | Estimated |
| | constant of lux promoter | | |
| | and [LuxR-3OC6HSL]₂ | | |
| | binding | | |
| K_{L} | Dissociation constant of | 1.6e−8 | Ref. 6 |
| | 3OC6HSL and LuxR binding | | |
| | (mol · L⁻¹) | | |
| K_{M} | Michaelis-Menten constant | 3 | Estimated |
| | of translation process | | |
| n | cooperativity of 3OC6HSL- | 1.3 | Ref. 6 |
| | LuxR binding | | |

Mathematic modeling and data fitting for cell-free gene expression analysis. When analyzing in vitro experiments, we used a mathematical model to help interpret results. Modeling of transcription and translation steps can take different forms because of different levels of details considered. A simple model only considers them as linear production and degradation^{6,8}. Some models use Michaelis-Menten or Hill-function-like terms to describe production or degradation processes, to account for nonlinear bottleneck or saturation effects due to the limitations of cellular machineries^{5,9,10}. Since the cell free system provides abundant molecular machinery for transcription and translation, we chose to use a simplified model that includes only transcription and translation steps without nonlinear terms. There are only four parameters in our simplified model. Two production rates can be freely scaled to fit experimental results and the protein degradation rate is also fixed according to literature reported values. The parameter studied in detail is the RNA degradation rate, which is directly related to different versions of dtRNAs used in each experiment.

Translation and transcription in vitro can be described by a simple 2D ordinary differential equation (ODE):

$$\frac{dM}{dt} = α - β M \\ \frac{dP}{dt} = γ M - δ P$$

where M stands for mRNA abundance and P stands for GFP abundance over time. α, β, γ, and δ are the mRNA production rate, mRNA degradation rate, GFP translation rate, and GFP degradation rate, respectively. This simple ODE can be solved analytically and give us the expression of GFP abundance over time:

$$GFP ( t ) = P ( t ) = \frac{αγ}{βδ} ( 1 - \frac{β}{β - δ} {e}^{- δ t} - \frac{δ}{δ - β} {e}^{- β t} )$$

Using this formula, we can fit time series data in FIG. 4b. In this fitting, α and γ are fixed to represent scale of fluorescence measurement and will not affect temporal dynamics (derivation below). δ is fixed to be 0.05 min⁻¹, consistent with reported in vitro half-life of GFP to be around 14 mintues¹¹. β, the only parameter that is changing over different experiments due to dtRNA engineering, is fitted against the data using lsqcurvefit from Matlab. Without inhibitor, β is ˜0.95 min⁻¹ for control, and around ˜0.15 min⁻¹ for dtRNA. With inhibitor, β is ˜0.18 min⁻¹ for control, and around ˜0.07 min⁻¹ for dtRNAs. Even though these values are only rough estimates of these molecule's chemical property in vitro. They still corroborate with experimental observations, showing decreased degradation rate for dtRNA and for cases with RNase inhibitor applied.

After fitting, the equation

$$Rate ( t ) = {GFP}^{′} ( t ) = \frac{αγ}{β - δ} ( {e}^{- δ t} - {e}^{- β t} )$$

is used to compute GFP accumulation rate over time (FIG. 4c) to show its dynamics. One easily recognizable feature of the GFP accumulation rate curve is its peak, which occurs when the curve's derivative equals to zero. We know from the equation above that

$${Rate}^{′} ( t ) = \frac{αγ}{β - δ} ( β {e}^{- β t} - δ {e}^{- δ t} )$$

So when

$$t = \frac{ln ( β ) - ln ( δ )}{β - δ} ,$$

Rate′(t)=0 and Rate(t) reaches its maximal value. As we can see, the location of the peak only depends on degradation rates. When protein degradation remains constant, the only factor affecting the peak locations is mRNA degradation rate, which is being tuned by dtRNA. As the mRNA getting more stable, β decreases and the curve peak shifts to the right.

Mathematic modeling and data fitting for cell-free RNA expression analysis. We used a simple mathematical model to calculate the RNA half-life and to interpret the results of in vitro RNA expression with data fitting. During the experiment, RNAs are generated through transcription and digested through degradation. We describe this RNA expression process with following ordinary differential equation:

$$\frac{dR}{dt} = v - δ R \qquad [ Eq4 ]$$

R stands for RNA concentration, which is indicated by the value of fluorescence measurements. v is the transcription rate which can vary in a relatively small range due to the variation of sequence. δ is the degradation rate, which is affected by dtRNAs. We then analytically solved this simple ODE and obtained the function of RNA concentration over time:

$$R ( t ) = \frac{v}{δ} + ( {R}_{0} - \frac{v}{δ} {e}^{- δ t} ) \qquad [ Eq5 ]$$

where R₀ is the initial RNA concentration. With this formula, we can fit RNA expression data of all 64 dtRNAs and control. In our fitting, we set a small boundary, 10±4 min⁻¹, for v, since this transcription rate is affected by the variation of dtRNA sequence. δ is the main parameter changing over different dtRNA designs. Also, considering the error caused by the initial small value of experimental data, we used the mean value of first three data points as the initial RNA concentration R₀. We fitted v and δ against experimental data using lsqcurvefit from Matlab. Then we calculated RNA concentration over time with Eq5 and RNA half-life with the formula:

$${t}_{1 / 2} = \frac{ln 2}{δ} \qquad [ Eq6 ]$$

As shown in Table 5, most of RNA with dtRNAs (45/64) has longer half-life than control.

### MAIN REFERENCES

1. Cambray, G., Guimaraes, J. C. & Arkin, A. P. Evaluation of 244,000 synthetic sequences reveals design principles to optimize translation in Escherichia coli. Nat Biotechnol 36, 1005-1015 (2018).
2. Mutalik, V. K. et al. Precise and reliable gene expression via standard transcription and translation initiation elements. Nat Methods 10, 354-360 (2013).
3. Michaels, Y. S. et al. Precise tuning of gene expression levels in mammalian cells. Nat Commun 10, 818 (2019).
4. Delivering the promise of RNA therapeutics. Nat Med 25, 1321-1321 (2019).
5. Kole, R., Krainer, A. R. & Altman, S. RNA therapeutics: beyond RNA interference and antisense oligonucleotides. Nat Rev Drug Discov 11, 125-140 (2012).
6. Pardee, K. et al. Rapid, Low-Cost Detection of Zika Virus Using Programmable Biomolecular Components. Cell 165, 1255-1266 (2016).
7. Slomovic, S., Pardee, K. & Collins, J. J. Synthetic biology devices for in vitro and in vivo diagnostics. Proc. Natl. Acad. Sci. U.S.A. 112, 14429-14435 (2015).
8. Chen, Y. Y., Galloway, K. E. & Smolke, C. D. Synthetic biology: advancing biological frontiers by building synthetic systems. Genome Biol. 13, 240 (2012).
9. Khalil, A. S. & Collins, J. J. Synthetic biology: applications come of age. Nat. Rev. Genet. 11, 367-379 (2010).
10. Hammer, K., Mijakovic, I. & Jensen, P. R. Synthetic promoter libraries—tuning of gene expression. Trends Biotechnol. 24, 53-55 (2006).
11. Ellis, T., Wang, X. & Collins, J. J. Diversity-based, model-guided construction of synthetic gene networks with predicted functions. Nat. Biotechnol. 27, 465-471 (2009).
12. Siegl, T., Tokovenko, B., Myronovskyi, M. & Luzhetskyy, A. Design, construction and characterisation of a synthetic promoter library for fine-tuned gene expression in actinomycetes. Metab. Eng. 19, 98-106 (2013).
13. de Smit, M. H. & van Duin, J. Translational initiation on structured messengers. Another role for the Shine-Dalgarno interaction. J. Mol. Biol. 235, 173-184 (1994).
14. Salis, H. M., Mirsky, E. A. & Voigt, C. A. Automated design of synthetic ribosome binding sites to control protein expression. Nat. Biotechnol. 27, 946-950 (2009).
15. de Smit, M. H. & van Duin, J. Control of translation by mRNA secondary structure in Escherichia coli. A quantitative analysis of literature data. J. Mol. Biol. 244, 144-150
16. Oesterle, S., Gerngross, D., Schmitt, S., Roberts, T. M. & Panke, S. Efficient engineering of chromosomal ribosome binding site libraries in mismatch repair proficient Escherichia coli. Sci Rep 7, 12327 (2017).
17. Chen, Y.-J. et al. Characterization of 582 natural and synthetic terminators and quantification of their design constraints. Nat. Methods 10, 659-664 (2013).
18. Gardner, P. P., Barquist, L., Bateman, A., Nawrocki, E. P. & Weinberg, Z. RNIE: genome-wide prediction of bacterial intrinsic terminators. Nucleic Acids Res. 39, 5845-5852 (2011).
19. Kingsford, C. L., Ayanbule, K. & Salzberg, S. L. Rapid, accurate, computational discovery of Rho-independent transcription terminators illuminates their relationship to DNA uptake. Genome Biol. 8, R22 (2007).
20. Green, A. A., Silver, P. A., Collins, J. J. & Yin, P. Toehold Switches: De-Novo-Designed Regulators of Gene Expression. Cell 159, 925-939 (2014).
21. Chappell, J., Takahashi, M. K. & Lucks, J. B. Creating small transcription activating RNAs. Nat. Chem. Biol. 11, 214-220 (2015).
22. Green, A. A. et al. Complex cellular logic computation using ribocomputing devices. Nature 548, 117-121 (2017).
23. Kim, J. et al. De novo-designed translation-repressing riboregulators for multi-input cellular logic. Nat Chem Biol (2019) doi:10.1038/s41589-019-0388-1.
24. Hoynes-O'Connor, A., Hinman, K., Kirchner, L. & Moon, T. S. De novo design of heat-repressible RNA thermosensors in E. coli. Nucleic Acids Res. 43, 6166-6179 (2015).
25. Lou, C., Stanton, B., Chen, Y.-J., Munsky, B. & Voigt, C. A. Ribozyme-based insulator parts buffer synthetic circuits from genetic context. Nat. Biotechnol. 30, 1137-1142 (2012).
26. Qi, L. S. et al. Repurposing CRISPR as an RNA-guided platform for sequence-specific control of gene expression. Cell 152, 1173-1183 (2013).
27. Matharu, N. et al. CRISPR-mediated activation of a promoter or enhancer rescues obesity caused by haploinsufficiency. Science 363, (2019).
28. Hawkins, J. S., Wong, S., Peters, J. M., Almeida, R. & Qi, L. S. Targeted Transcriptional Repression in Bacteria Using CRISPR Interference (CRISPRi). Methods Mol. Biol. 1311, 349-362 (2015).
29. Cleto, S., Jensen, J. V., Wendisch, V. F. & Lu, T. K. Corynebacterium glutamicum Metabolic Engineering with CRISPR Interference (CRISPRi). ACS Synth Biol 5, 375-385
30. Siu, K.-H. & Chen, W. Riboregulated toehold-gated gRNA for programmable CRISPR-Cas9 function. Nat Chem Biol 15, 217-220 (2019).
31. Oesinghaus, L. & Simmel, F. C. Switching the activity of Cas12a using guide RNA strand displacement circuits. Nat Commun 10, 2092 (2019).
32. Hanewich-Hollatz, M. H., Chen, Z., Hochrein, L. M., Huang, J. & Pierce, N. A. Conditional Guide RNAs: Programmable Conditional Regulation of CRISPR/Cas Function in Bacterial and Mammalian Cells via Dynamic RNA Nanotechnology. ACS Cent. Sci. 5, 1241-1249 (2019).
33. Wu, F., Zhang, Q. & Wang, X. Design of Adjacent Transcriptional Regions to Tune Gene Expression and Facilitate Circuit Construction. Cell Syst 6, 206-215.e6 (2018).
34. Pfleger, B. F., Pitera, D. J., Smolke, C. D. & Keasling, J. D. Combinatorial engineering of intergenic regions in operons tunes expression of multiple genes. Nat. Biotechnol. 24, 1027-1032 (2006).
35. Leppek, K., Das, R. & Barna, M. Functional 5′ UTR mRNA structures in eukaryotic translation regulation and how to find them. Nat. Rev. Mol. Cell Biol. 19, 158-174 (2018).
36. Win, M. N. & Smolke, C. D. Higher-Order Cellular Information Processing with Synthetic RNA Devices. Science 322, 456-460 (2008).
37. Win, M. N. & Smolke, C. D. A modular and extensible RNA-based gene-regulatory platform for engineering cellular function. Proceedings of the National Academy of Sciences 104, 14283-14288 (2007).
38. Sastalla, I., Chim, K., Cheung, G. Y. C., Pomerantsev, A. P. & Leppla, S. H. Codon-optimized fluorescent proteins designed for expression in low-GC gram-positive bacteria. Appl. Environ. Microbiol. 75, 2099-2110 (2009).
39. Yang, T. T., Cheng, L. & Kain, S. R. Optimized codon usage and chromophore mutations provide enhanced sensitivity with the green fluorescent protein. Nucleic Acids Res. 24, 4592-4593 (1996).
40. Rauhut, R. & Klug, G. mRNA degradation in bacteria. FEMS Microbiol. Rev. 23, 353-370 (1999).
41. Hui, M. P., Foley, P. L. & Belasco, J. G. Messenger RNA degradation in bacterial cells. Annu. Rev. Genet. 48, 537-559 (2014).
42. Mackie, G. A. RNase E: at the interface of bacterial RNA processing and decay. Nat Rev Microbiol 11, 45-57 (2013).
43. Carpousis, A. J. The RNA degradosome of Escherichia coli: an mRNA-degrading machine assembled on RNase E. Annu. Rev. Microbiol. 61, 71-87 (2007).
44. Case, C. C., Simons, E. L. & Simons, R. W. The IS10 transposase mRNA is destabilized during antisense RNA control. EMBO J. 9, 1259-1266 (1990).
45. Arnold, T. E., Yu, J. & Belasco, J. G. mRNA stabilization by the ompA 5′ untranslated region: two protective elements hinder distinct pathways for mRNA degradation. RNA 4, 319-330 (1998).
46. Bouvet, P. & Belasco, J. G. Control of RNase E-mediated RNA degradation by 5′-terminal base pairing in E. coli. Nature 360, 488-491 (1992).
47. Emory, S. A., Bouvet, P. & Belasco, J. G. A 5′-terminal stem-loop structure can stabilize mRNA in Escherichia coli. Genes & Development 6, 135-148 (1992).
48. Carrier, T. A. & Keasling, J. D. Library of synthetic 5′ secondary structures to manipulate mRNA stability in Escherichia coli. Biotechnol. Prog. 15, 58-64 (1999).
49. Cetnar, D. P. & Salis, H. M. Systematic Quantification of Sequence and Structural Determinants Controlling mRNA stability in Bacterial Operons. ACS Synth. Biol. acssynbio.0c00471 (2021) doi:10.1021/acssynbio.0c00471.
50. Viegas, S. C., Apura, P., Martinez-Garcia, E., de Lorenzo, V. & Arraiano, C. M. Modulating Heterologous Gene Expression with Portable mRNA-Stabilizing 5′-UTR Sequences. ACS Synth Biol 7, 2177-2188 (2018).
51. Espah Borujeni, A., Channarasappa, A. S. & Salis, H. M. Translation rate is controlled by coupled trade-offs between site accessibility, selective RNA unfolding and sliding at upstream standby sites. Nucleic Acids Res. 42, 2646-2659 (2014).
52. Gusarov, I. & Nudler, E. The mechanism of intrinsic transcription termination. Mol. Cell 3, 495-504 (1999).
53. d′Aubenton Carafa, Y., Brody, E. & Thermes, C. Prediction of rho-independent Escherichia coli transcription terminators. A statistical analysis of their RNA stem-loop structures. J. Mol. Biol. 216, 835-858 (1990).
54. Varani, G. Exceptionally stable nucleic acid hairpins. Annu Rev Biophys Biomol Struct 24, 379-404 (1995).
55. Caron, M.-P. et al. Dual-acting riboswitch control of translation initiation and mRNA decay. Proc. Natl. Acad. Sci. U.S.A. 109, E3444-3453 (2012).
56. Wu, F., Menn, D. J. & Wang, X. Quorum-sensing crosstalk-driven synthetic circuits: from unimodality to trimodality. Chem. Biol. 21, 1629-1638 (2014).
57. Ng, W.-L. & Bassler, B. L. Bacterial quorum-sensing network architectures. Annu. Rev. Genet. 43, 197-222 (2009).
58. Wu, M. et al. Engineering of regulated stochastic cell fate determination. Proc. Natl. Acad. Sci. U.S.A. 110, 10610-10615 (2013).
59. Gardner, T. S., Cantor, C. R. & Collins, J. J. Construction of a genetic toggle switch in Escherichia coli. Nature 403, 339-342 (2000).
60. Pardee, K. et al. Paper-based synthetic gene networks. Cell 159, 940-954 (2014).
61. Karzbrun, E., Tayar, A. M., Noireaux, V. & Bar-Ziv, R. H. Synthetic biology. Programmable on-chip DNA compartments as artificial cells. Science 345, 829-832 (2014).
62. Dudley, Q. M., Karim, A. S. & Jewett, M. C. Cell-free metabolic engineering: biomanufacturing beyond the cell. Biotechnol J 10, 69-82 (2015).
63. Ma, D., Shen, L., Wu, K., Diehnelt, C. W. & Green, A. A. Low-cost detection of norovirus using paper-based cell-free systems and synbody-based viral enrichment. Synth Biol (Oxf) 3, ysy018 (2018).
64. Jeong, D. et al. Cell-Free Synthetic Biology Platform for Engineering Synthetic Biological Circuits and Systems. Methods Protoc 2, (2019).
65. Huang, A. et al. BioBits™ Explorer: A modular synthetic biology education kit. Sci Adv 4, eaat5105 (2018).
66. Russell, J. B. The energy spilling reactions of bacteria and other organisms. J. Mol. Microbiol. Biotechnol. 13, 1-11 (2007).
67. Ceroni, F. et al. Burden-driven feedback control of gene expression. Nat. Methods 15, 387-393 (2018).
68. Kwon, Y.-C. & Jewett, M. C. High-throughput preparation methods of crude extract for robust cell-free protein synthesis. Sci Rep 5, 8663 (2015).
69. Lavickova, B. & Maerkl, S. J. A Simple, Robust, and Low-Cost Method To Produce the PURE Cell-Free System. ACS Synth. Biol. 8, 455-462 (2019).
70. Silverman, A. D., Kelley-Loughnane, N., Lucks, J. B. & Jewett, M. C. Deconstructing Cell-Free Extract Preparation for in Vitro Activation of Transcriptional Genetic Circuitry. ACS Synth Biol 8, 403-414 (2019).
71. Wu, F., Su, R.-Q., Lai, Y.-C. & Wang, X. Engineering of a synthetic quadrastable gene network to approach Waddington landscape and cell fate determination. Elife 6, (2017).
72. Zadeh, J. N. et al. NUPACK: Analysis and design of nucleic acid systems. J Comput Chem 32, 170-173 (2011).
73. Standage-Beier, K., Zhang, Q. & Wang, X. Targeted Large-Scale Deletion of Bacterial Genomes Using CRISPR-Nickases. ACS Synth Biol 4, 1217-1225 (2015).
74. Gibson, D. G. et al. Enzymatic assembly of DNA molecules up to several hundred kilobases. Nat. Methods 6, 343-345 (2009).

### SUPPLEMENTAL REFERENCES

1. Zadeh, J. N. et al. NUPACK: Analysis and design of nucleic acid systems. J Comput Chem 32, 170-173 (2011).
2. Kudla, G., Murray, A. W., Tollervey, D. & Plotkin, J. B. Coding-sequence determinants of gene expression in Escherichia coli. Science 324, 255-258 (2009).
3. Wu, F., Zhang, Q. & Wang, X. Design of Adjacent Transcriptional Regions to Tune Gene Expression and Facilitate Circuit Construction. Cell Syst 6, 206-215.e6 (2018).
4. Serra, M. J. & Turner, D. H. Predicting thermodynamic properties of RNA. Meth. Enzymol. 259, 242-261 (1995).
5. Basu, S., Gerchman, Y., Collins, C. H., Arnold, F. H. & Weiss, R. A synthetic multicellular system for programmed pattern formation. Nature 434, 1130-1134 (2005).
6. Wu, F., Menn, D. J. & Wang, X. Quorum-sensing crosstalk-driven synthetic circuits: from unimodality to trimodality. Chem. Biol. 21, 1629-1638 (2014).
7. Ermentrout, B. Xppaut. In: Computational Systems Neurobiology, Springer Science+Business Media, Dordrecht, pp. 519-531 (2012).
8. Gardner, T. S., Cantor, C. R. & Collins, J. J. Construction of a genetic toggle switch in Escherichia coli. Nature 403, 339-342 (2000).
9. Wu, F., Su, R.-Q., Lai, Y.-C. & Wang, X. Engineering of a synthetic quadrastable gene network to approach Waddington landscape and cell fate determination. Elife 6, (2017).
10. Zhang, R. et al. Topology-dependent interference of synthetic gene circuit function by growth feedback. Nat. Chem. Biol. (2020) doi:10.1038/s41589-020-0509-x.
11. Shin, J. & Noireaux, V. Study of messenger RNA inactivation and protein degradation in an Escherichia coli cell-free expression system. J Biol Eng 4, 9 (2010).

We claim:

1. A degradation tuning RNA (dtRNA) comprising the following components, ordered from 5′ to 3′:
a) a leader sequence comprising zero to six nucleotides,
b) a first stem-forming region,
c) a loop-forming region comprising at least three nucleotides,
d) a second stem-forming region, and
e) an insulator sequence comprising at least five nucleotides;
wherein the first stem-forming region and the second stem-forming region form a stem that is at three nucleotides in length.

2. The dtRNA of claim 1, wherein the insulator sequence is single stranded.

3. The dtRNA of claim 1, wherein the dtRNA comprises a sequence selected from SEQ ID NO:1-82.

4. A method of modulating the stability of an RNA, the method comprising:
a) forming the dtRNA of claim 1; and
b) inserting the dtRNA into the RNA in a position that is 5′ to the functional portion of the RNA.

5. The method of claim 4, wherein the GC content of the dtRNA is between about 40% to about 80%.

6. (canceled)

7. The method of claim 4, wherein the stem is about 8 to about 15 base pairs in length.

8. (canceled)

9. The method of claim 4, wherein the loop-forming region is between about three nucleotides and about 30 nucleotides in length.

10. (canceled)

11. The method of claim 4, wherein the leader sequence is less than 18 nucleotides in length.

12. The method of claim 4, wherein the leader sequence at least 18 nucleotides in length.

13. The method of claim 4, wherein the dtRNA comprises one or more RNase E cleavage sites.

14. The method of claim 4, wherein insertion of the dtRNA increases the stability of the RNA.

15. The method of claim 4, wherein insertion of the dtRNA decreases the stability of the RNA.

16. The method of claim 4, wherein the RNA is an mRNA and the dtRNA is inserted between the transcription start site and the ribosome binding site of a DNA molecule encoding the mRNA.

17. The method of claim 16, wherein insertion of the dtRNA increases the expression of a protein encoded by the mRNA.

18. The method of claim 16, wherein the mRNA is part of a synthetic gene regulatory circuit.

19. The method of claim 4, wherein the RNA is a noncoding RNA.

20. The method of claim 19, wherein the noncoding RNA is part of a CRISPR-based system.

21. The method of claim 19, wherein the noncoding RNA comprises a toehold switch.

22. The method of claim 4, wherein the RNA is expressed in a cell-free expression system.

23. A DNA construct comprising a promoter that is operably connected to a sequence encoding the dtRNA of claim 1 and a multi-cloning site or a functional RNA.

24. (canceled)
